(19) United States
(12) Patent Application Publication
(10) Pub. No.: US 2017/0246269 A1
(43) Pub. Date: Aug. 31, 2017

(54) METHODS OF TREATING OR PREVENTING PERIODONTITIS AND DISEASES ASSOCIATED WITH PERIODONTITIS

(71) Applicants: Georgios Hajishengallis, Philadelphia, PA (US); John D. Lambris, Philadelphia, PA (US)

(72) Inventors: Georgios Hajishengallis, Philadelphia, PA (US); John D. Lambris, Philadelphia, PA (US)

(21) Appl. No.: 15/457,660

(22) Filed: Mar. 13, 2017

Related U.S. Application Data
(63) Continuation of application No. 14/487,396, filed on Sep. 16, 2014, now abandoned.
Application No. 14/487,396 is a continuation of application No. 13/574,542, filed on Sep. 20, 2012, now abandoned.
(60) Provisional application No. 61/297,535, filed on Jan. 22, 2010.
(60) Provisional application No. 61/418,218, filed on Nov. 30, 2010.

(51) Int. Cl. A61K 38/57; A61K 9/00; G01N 33/68; A61K 38/17

(52) U.S. Cl. CPC A61K 38/57; A61K 38/1709; A61K 9/0063; G01N 33/6893; G01N 2800/18

(57) ABSTRACT

The present disclosure describes methods for preventing or treating periodontitis or diseases associated with periodontitis. The present disclosure also describes methods of screening for compounds that can be used to prevent or treat periodontitis or diseases associated with periodontitis.

### CROSS REFERENCE TO RELATED APPLICATIONS

This application claims benefit under 35 U.S.C. 119(e) to U.S. Application No. 61/297,535 filed on Jan. 22, 2010 and U.S. Application No. 61/418,218 filed on Nov. 30, 2010. Both applications are incorporated herein in their entirety.

### FEDERALLY SPONSORED RESEARCH OR DEVELOPMENT

This invention was made with government support under Grant Nos. GM-62134, AI-068730, DE015254, DE021580, DE017138, and DE018292 awarded by U.S. Public Health Service. The government has certain rights in the invention.

### TECHNICAL FIELD

This disclosure generally relates to periodontal disease and methods of treating or preventing periodontitis.

### BACKGROUND

Although traditionally perceived as an antimicrobial enzyme system in serum, complement is now recognized as a central component of host defense impacting both innate and adaptive immunity. More recently, complement was suggested to crosstalk with another major innate defense system, the Toll-like receptors (TLRs), to coordinate the host response to infection. Not surprisingly, given its importance in fighting pathogens, complement constitutes a key target of immune evasion by microbes that cause persistent infections. The present disclosure describes a novel strategy of immune subversion used by P. gingivalis, which can be exploited to treat or prevent periodontitis and diseases associated with periodontitis.

### SUMMARY

The present disclosure describes methods for preventing or treating periodontitis or diseases associated with periodontitis. The present disclosure also describes methods of screening for compounds that can be used to prevent or treat periodontitis or diseases associated with periodontitis.

In one aspect, a method of treating or preventing periodontitis or diseases associated with periodontitis in an individual is provided. Such a method generally includes administering a compound to the individual that inhibits or blocks C5a receptor expression, activity, or activation. In one embodiment, the compound is selected from the group consisting of acetylated phenylalanine-(ornithine-proline-(D)cyclohexylalanine-tryptophan-arginine), W-54011, ADC-1004, CGS 32359, NDT9520492, NGD 2000-1, and NDT 9513727. In another embodiment, the compound is an antibody against the C5a receptor. In yet another embodiment, the compound is a peptidomimetic antagonist of the C5a receptor. Representative diseases associated with periodontitis include, without limitation, atherosclerosis, diabetes, and pre-term labor.

In another aspect, a method of treating or preventing periodontitis or diseases associated with periodontitis in an individual is provided. Such a method generally includes administering a compound to the individual that inhibits or blocks TLR2 expression or activity.

In still another aspect, a method of reducing the amount of Porphyromonas gingivalis and/or the inflammation caused by P. gingivital in an individual is provided. Generally, such a method includes administering, to the individual, a compound that inhibits or blocks C5a receptor expression, activity, or activation or a compound that inhibits or blocks TRL2 expression or activity. Representative compounds that inhibit or block C5a receptor expression, activity, or activation are described herein.

In still another aspect, a method of screening for compounds that treat or prevent periodontitis or diseases associated with periodontitis is provided. Such methods generally include contacting a cell, in the presence of P. gingivalis, with a test compound; and evaluating the cell for expression, activity, or activation of C5a receptor, expression or activity of TLR2, or crosstalk between C5a receptor and TLR2. Typically, a reduction in the expression, activity, or activation of C5a receptor, or a reduction in the expression or activity of TLR2, or a reduction in the crosstalk between C5a receptor and TLR2 in the presence of a test compound is indicative of a test compound that can be used to treat or prevent periodontitis or diseases associated with periodontitis. In certain embodiments, the cell is a mammalian cell. In certain embodiments, the cell is a recombinant cell comprising exogenous nucleic acids encoding C5a receptor and/or TLR2.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

### Part A: Microbial Hijacking of Complement-Toll-Like Receptor Crosstalk

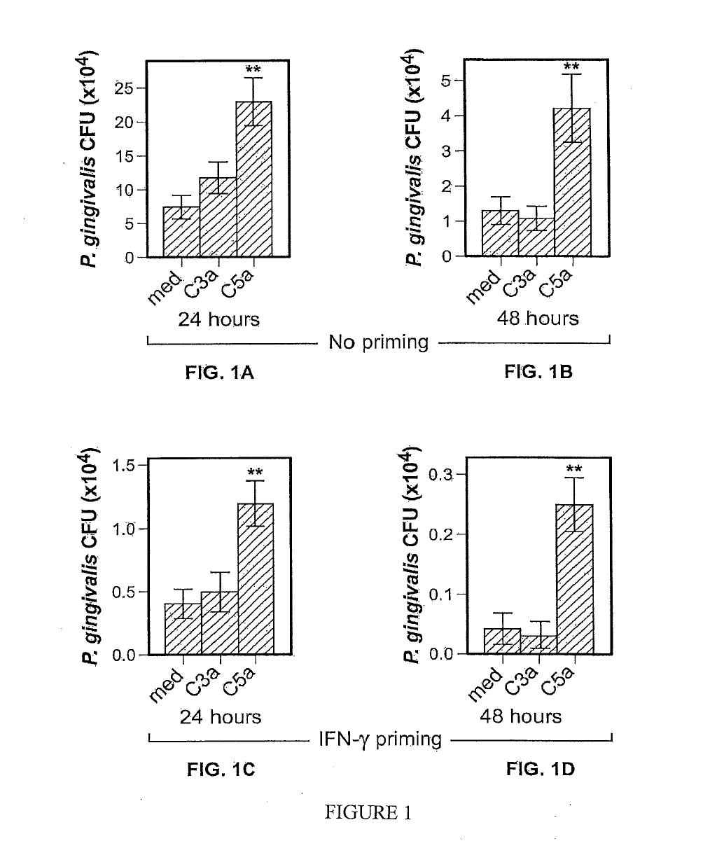

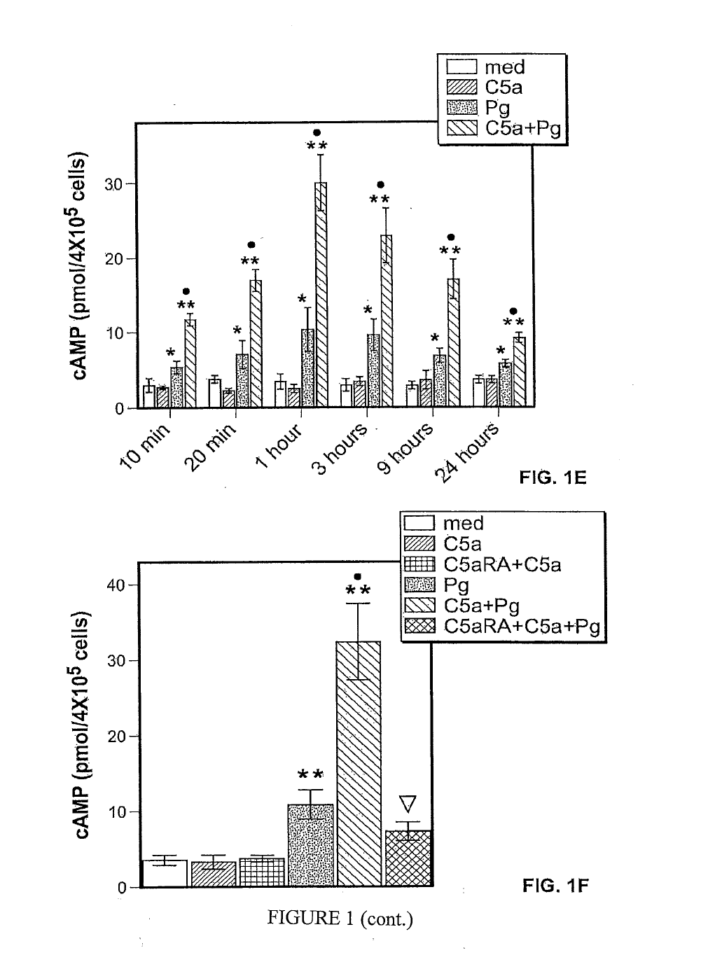

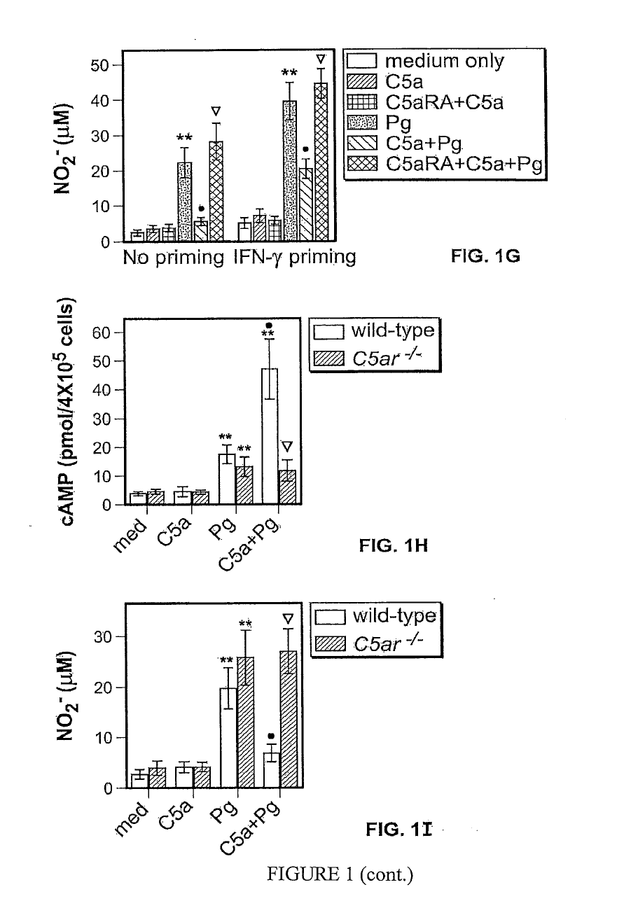

FIG. 1 demonstrates the immunosubversive effects of C5a on macrophages. (A-D) Peritoneal mouse macrophages were left untreated (A,B) or primed with 100 ng/ml IFN-γ (C,D) overnight, washed, and incubated with P. gingivalis (Pg; MOI=10:1) in the presence or absence of C3a (200 nM) or C5a (50 nM). Viable counts of internalized bacteria at 24 hours (A and C) or 48 hours (B and D) post-infection were determined by CFU enumeration. (E) Macrophages were incubated with medium only or with Pg in the presence or absence of C5a for the indicated times and assayed for induction of intracellular cAMP. (F) Similar experiment as in E, involving 1-hour incubation and the use of a specific C5a receptor antagonist (C5aRA; 1 μM), as indicated. (G) Unprimed or IFN-γ-primed macrophages were assayed for NO₂⁻ after 24-hour incubation with or without Pg and/or C5a, which acted in the absence or presence of C5aRA. (H-I) Similar experiments for induction of cAMP (H) and NO₂⁻ (I) using macrophages from both wild-type and C5aR-deficient (C5ar^{-/-}) mice. Data are means±SD (n=3) from typical experiments performed three (A-D, F, G) or two (E, H-I) times yielding consistent results. *, P<0.05 and **, P<0.01 vs. medium (med) control treatments. P<0.01 in C5a+Pg vs. Pg alone. Inverted triangles indicate significant (P<0.01) reversal of C5a effects by C5aRA or C5aR deficiency.

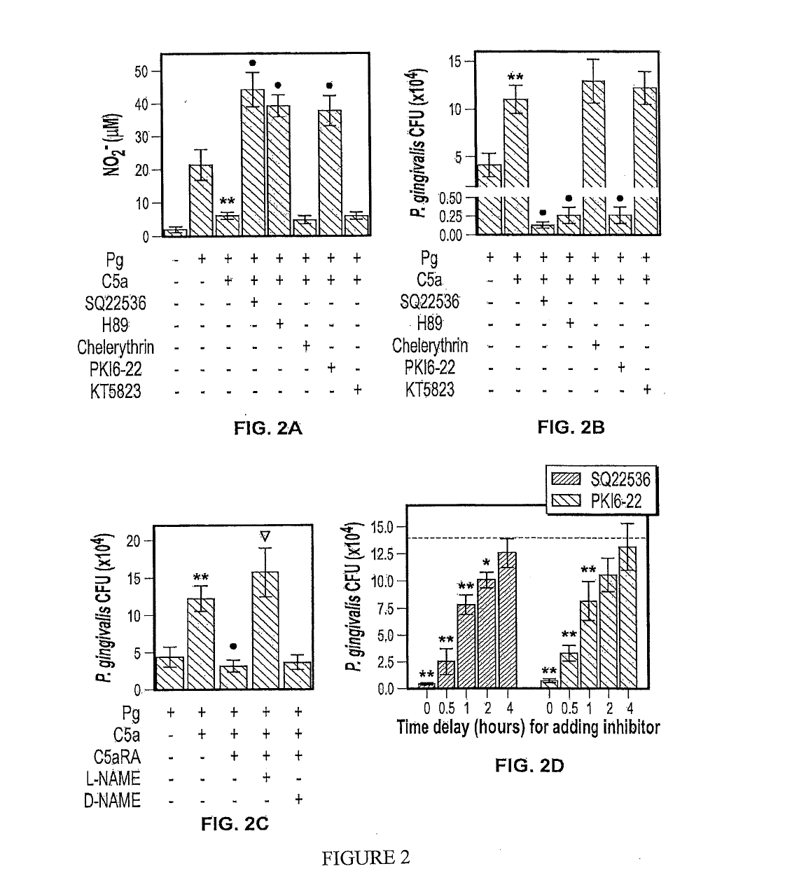

FIG. 2 demonstrates the C5a-mediated inhibition of nitric oxide and that promotion of P. gingivalis survival is cAMP- and PKA-dependent. (A and B) Mouse macrophages were pretreated or not with SQ22536 (cAMP synthesis inhibitor; 200 μM), H89 (PKA inhibitor; 5 chelerythrin (protein kinase C inhibitor; 5 μM), PKI 6-22 (peptide inhibitor of PKA; 1 μM), or KT5823 (peptide inhibitor of protein kinase G; 1 μM), and then infected with P. gingivalis (Pg; MOI=10:1) with or without C5a (50 nM), as indicated. (C) Macrophages were pretreated with 1 mM L-NAME (or D-NAME) and/or 1 μM C5aRA and then infected with Pg with or without C5a. (D) Macrophages were incubated with Pg and C5a in the absence or presence of SQ22536 or PKI 6-22, added prior to Pg and C5a (“0 time delay”) or with increasing delay times, as indicated. NO₂⁻ production (A) and viable counts of internalized bacteria (B-D) were determined at 24 hours postinfection. In D, the dashed line indicates Pg CFU in the absence of inhibitors (13.7±2.7[×10⁴] CFU). Results are means±SD (n=3) from typical experiments performed at least twice with consistent results. *, P<0.05 and **, P<0.01 vs. corresponding controls. , P<0.01 in C5a+Pg plus inhibitor or antagonist vs. C5a+Pg only. In C, the inverted triangle shows significant (P<0.01) reversal of the C5aRA effect.

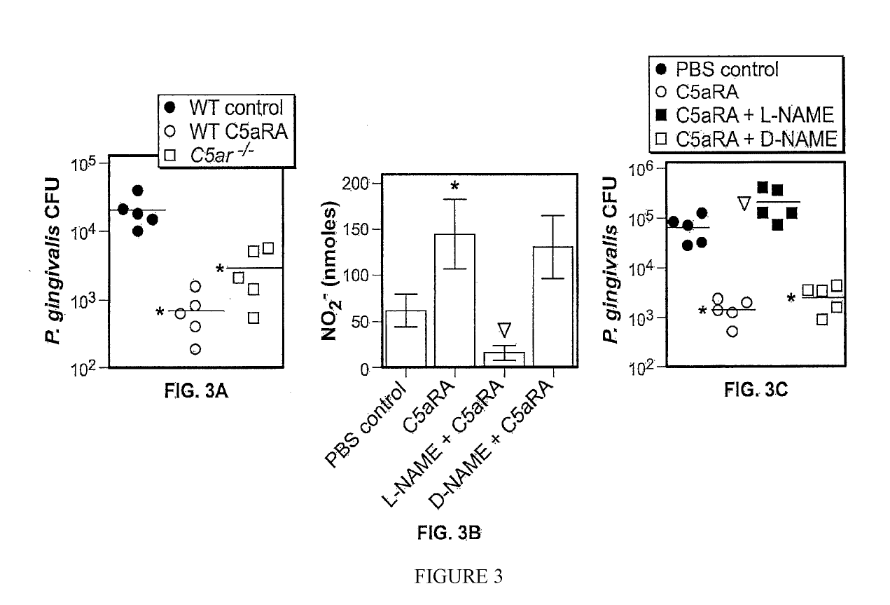

FIG. 3 demonstrates that P. gingivalis exploits C5aR signaling to inhibit nitric oxide production and promote its survival in vivo. (A) Wild-type (WT) mice were i.p. pretreated with C5aRA (1 mg/Kg body weight) or PBS control, followed by i.p. infection of these mice, as well as mice deficient in C5aR (C5a^{-/-}), with 5×10⁷ CFU P. gingivalis. (B and C) Wild-type mice were i.p. pretreated or not with C5aRA with or without L-NAME or D-NAME (0.1 ml of 12.5 mM solution, corresponding to 0.34 mg per mouse) followed by P. gingivalis i.p. infection. Peritoneal fluid was collected 24 hours postinfection and used to determine viable P. gingivalis CFU (A and C) and NO₂⁻ production (B). Data are from typical experiments performed twice yielding consistent findings and represent means±SD (n=5) or are shown for each individual mouse with horizontal lines denoting mean values. *, P<0.01 vs. controls. The inverted triangles show significant (P<0.01) reversal of the C5aRA effects.

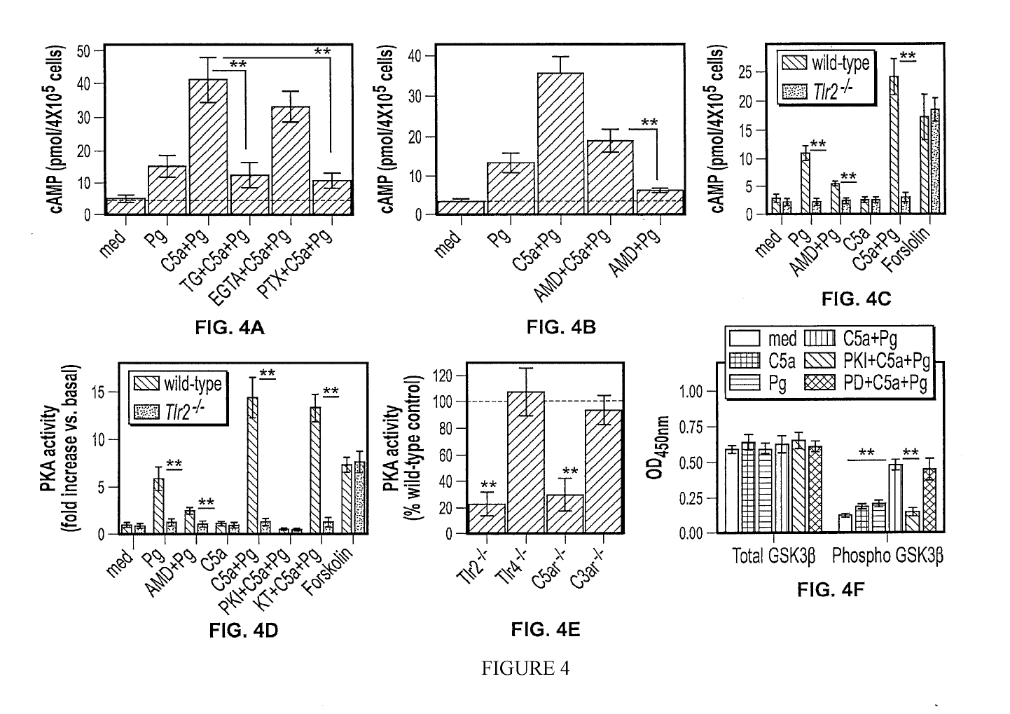

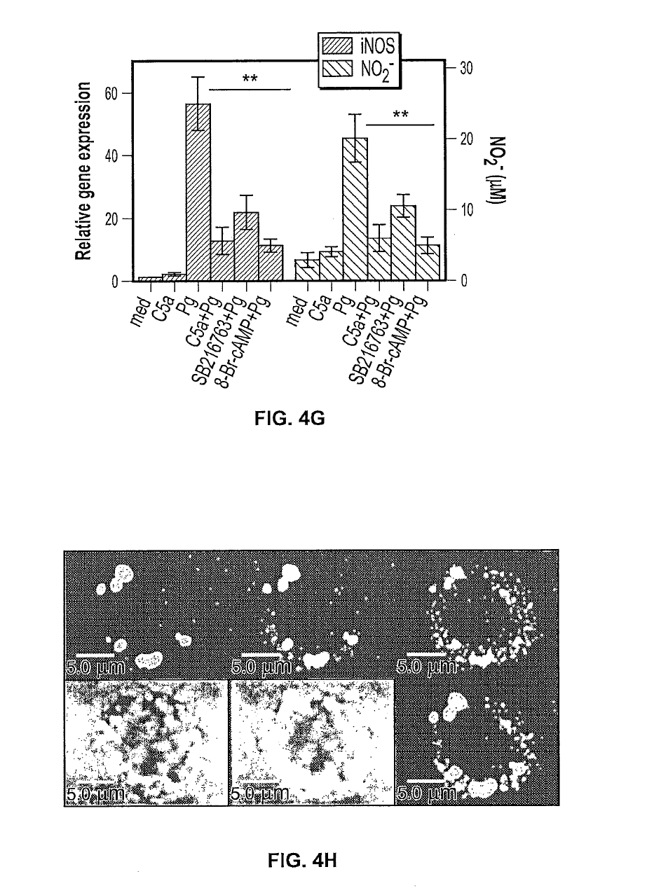

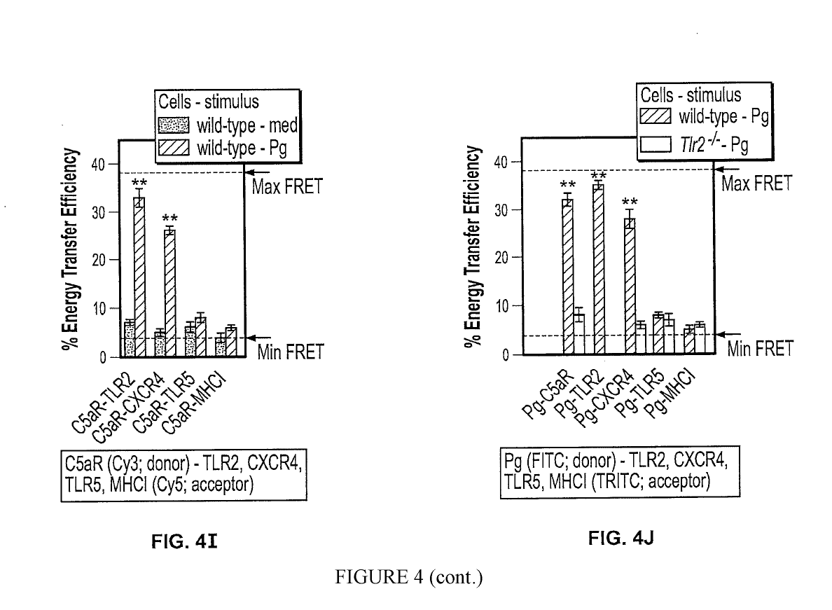

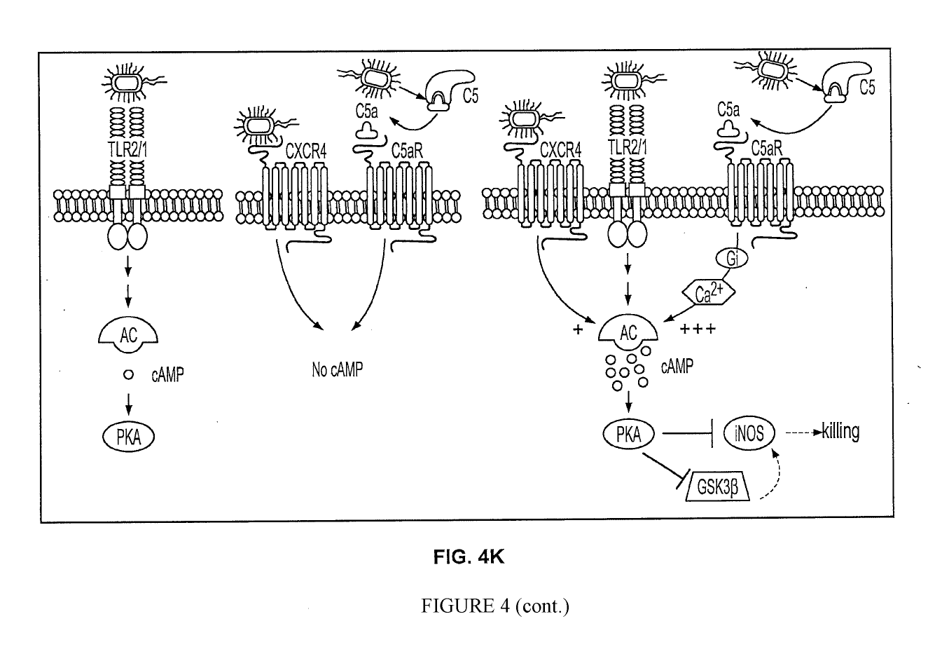

FIG. 4 demonstrates that the synergistic activation of the cAMP-PKA pathway requires C5aR-TLR2 crosstalk. Macrophages pretreated with 1 μM thapsigargin (TG), 5 mM EGTA, 100 ng/ml pertussis toxin (PTX) (A) or 1 μg/ml AMD3100 (B-D) were stimulated with P. gingivalis (Pg; MOI=10:1; 1 hour) with or without 50 nM C5a and assayed for cAMP (A-C) or PKA activity (D). PKA assay specificity was confirmed using PKI-6-22 and an irrelevant kinase inhibitor (KT5823). Forskolin (20 μM; 10-min) served as positive control in experiments with Tlr2^{-/-} macrophages (C and D). (E) PKA activities in freshly explanted peritoneal macrophages from Pg-infected mice (activities of indicated receptor-deficient cells expressed as % wild-type activity). (F) Macrophages pretreated with 1 μM PKI-6-22 or 25 μM PD98059 (PD; control) were stimulated with Pg, with or without C5a, and assayed for GSK3β Ser9-phosphorylation and total GSK3β. (G) Macrophages stimulated with Pg with or without C5a (50 nM), SB216763 (10 μM), or 8-Br-cAMP (100 μM) were assayed for iNOS expression (4 hours) or NO₂⁻ (24 hours). (H) Confocal colocalization of P. gingivalis (green), C5aR (red), and TLR2 (blue), as better shown in the bottom right merge image. (I) FRET between the indicated donors and acceptors measured from the increase in donor (Cy3 or FITC) fluorescence after acceptor (Cy5 or TRITC) photobleaching. Data are means±SD (n=3 except for E, n=5) from typical experiments performed at least twice with consistent results. *, P<0.05; **, P<0.01 between the indicated groups or vs. controls (E and I). (K) Pg induces weak TLR2-dependent cAMP induction (left), whereas CXCR4 or C5aR signaling alone fails to induce cAMP (middle). However, Pg-induced TLR2 signaling with concomitant activation of C5aR and, to a lesser extent, CXCR4 synergistically enhances the immunosuppressive cAMP-PKA pathway that inactivates GSK3β and impairs iNOS-dependent killing.

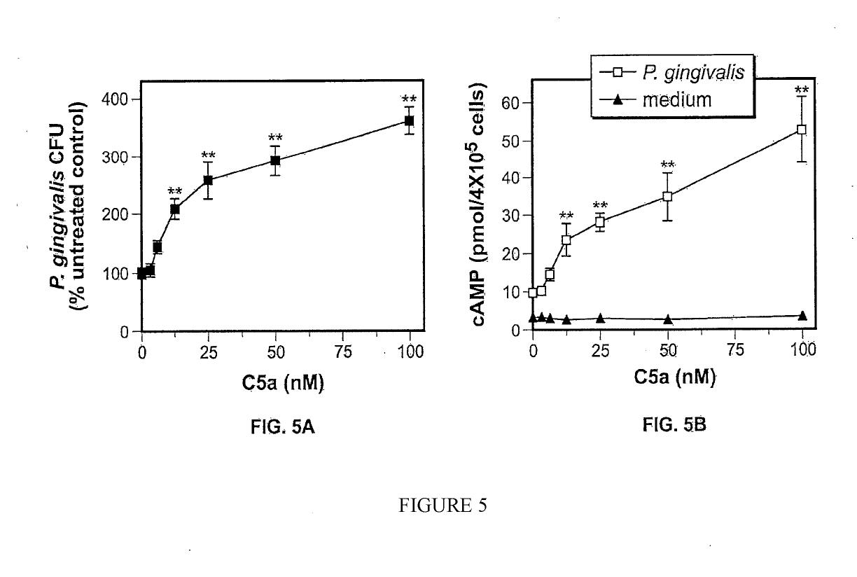

FIG. 5 are graphs showing that C5a dose-dependently promotes the intracellular survival of P. gingivalis and the cAMP response. Data are means±SD (n=3) from typical experiments, each performed twice yielding consistent results. ** P<0.01.

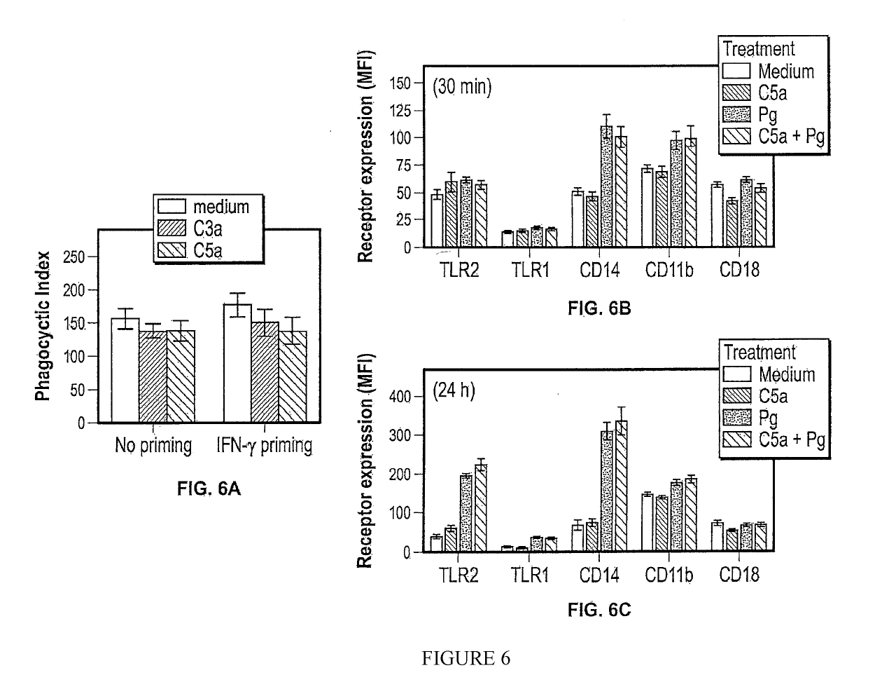

FIG. 6 is a graph showing that C5a does not affect P. gingivalis phagocytosis. Data are means±SD (n=3) from one of two independent sets of experiments yielding consistent results. MFI=mean fluorescent intensity.

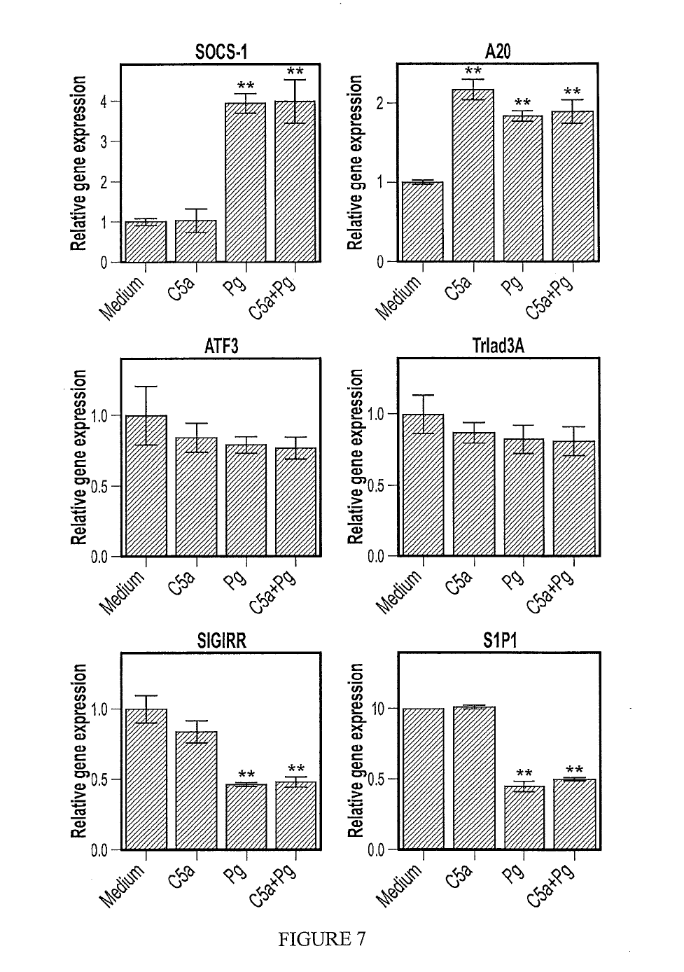

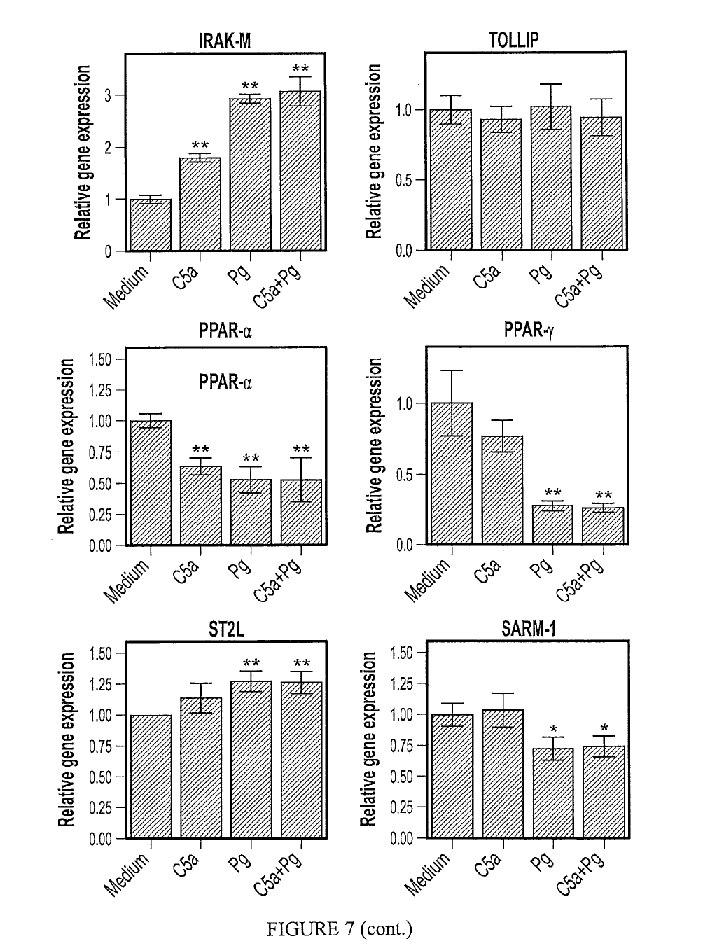

FIG. 7 is a graph showing the relative expression of negative regulators of TLR signaling in P. gingivalis-stimulated macrophages in the absence or presence of C5a. Results are shown as fold induction relative to medium-only-treated macrophages. Data are means±SD (n=3) from one of two independent sets of experiments yielding consistent results. *, P<0.05 and ** P<0.01 vs. medium-only control. SOCS-1, suppressor of cytokine signaling-1; IRAK-M, interleukin-1 receptor-associated kinase M; TOLLIP, Toll-interacting protein, ATF3, activating transcription factor-3; A20 is a ubiquitin-editing enzyme; Triad3A is an E3 ubiquitin-protein ligase; PPAR-α, peroxisome proliferative activated receptor-α; PPAR-γ, peroxisome proliferative activated receptor-γ; SIGIRR, single immunoglobulin interleukin-1-related receptor; S1P1, sphingosine 1-phosphate receptor type 1; ST2L is a type I transmembrane protein which sequesters MyD88 and MyD88 adaptor-like (Mal) protein; SARM-1, sterile alpha and HEAT/Armadillo motif protein-1.

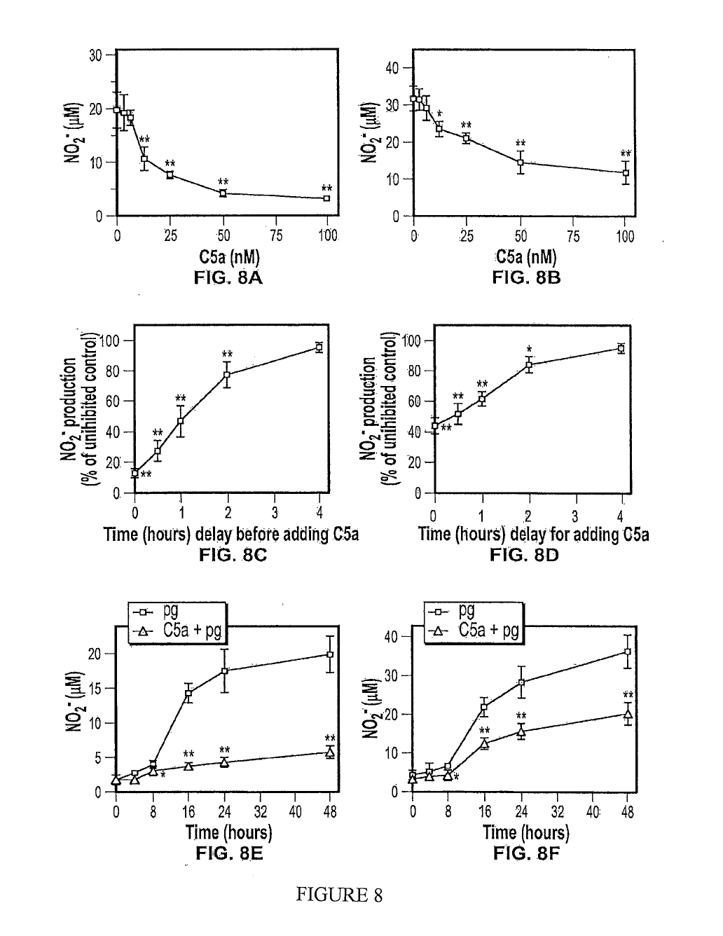

FIG. 8 demonstrates that C5a inhibits nitric oxide production in a dose- and time-dependent way. Data are means±SD (n=3) from typical experiments that were performed twice. Asterisks show significant (*, P<0.05; **, P<0.01) inhibition of NO₂⁻ production.

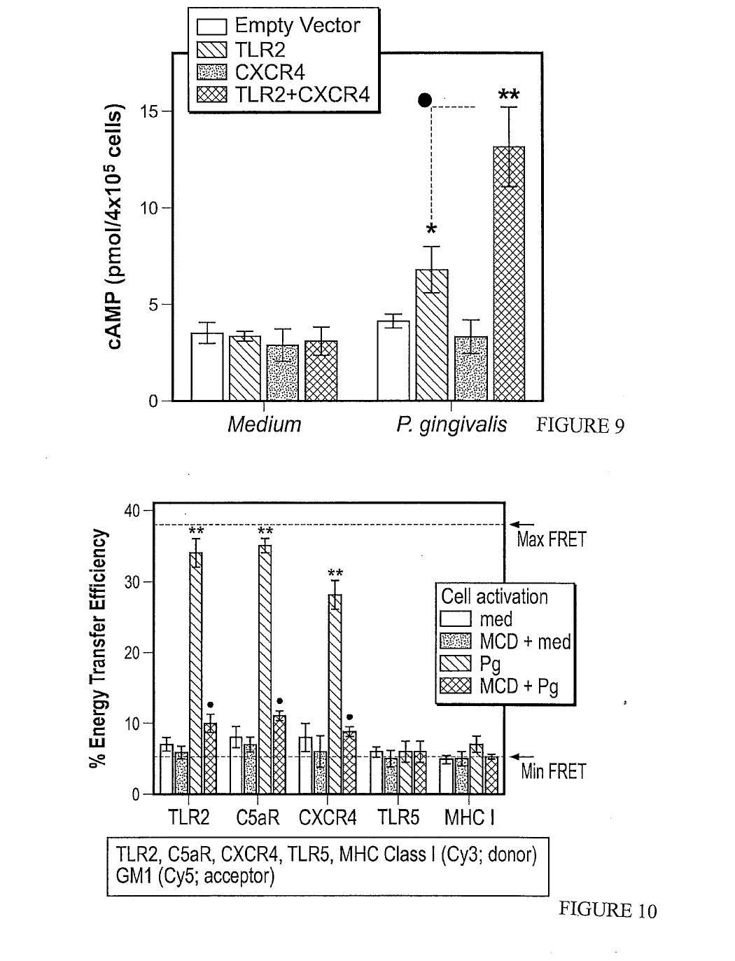

FIG. 9 shows the TLR2-dependent cAMP production by P. gingivalis. Data are means±SD (n=3) from a typical experiment performed three times. *, P<0.05 and **, P<0.01 vs. empty vector control. *, P<0.01 between the indicated groups.

FIG. 10 shows the association of TLR2, C5aR, and CXCR4 with GM1 (lipid raft marker) in P. gingivalis-stimulated macrophages. Data are means±SD (n=3). **, significant (P<0.01) FRET increase vs. medium-only control. *, significant (P<0.01) reversal of FRET increase by MCD.

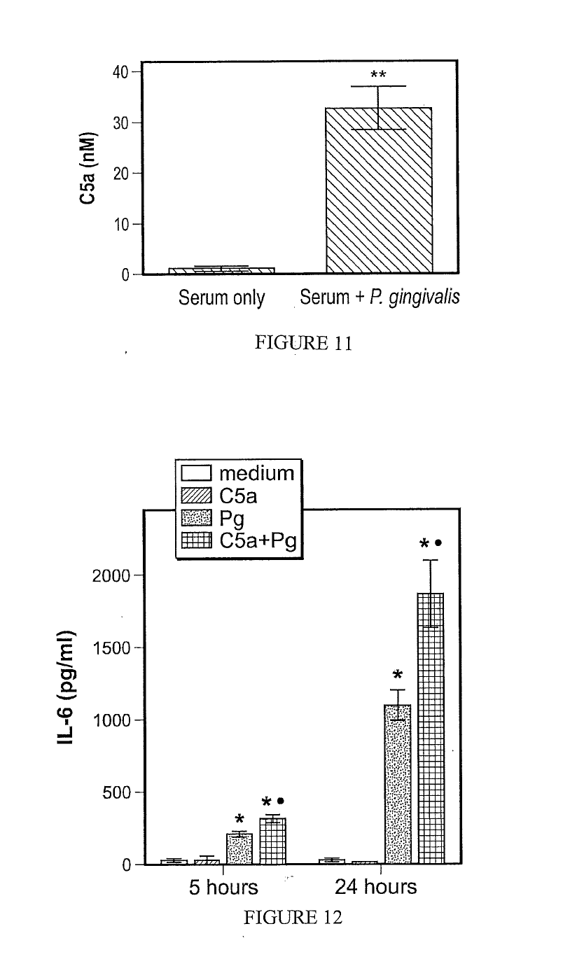

FIG. 11 shows the generation of C5a by P. gingivalis from heat-inactivated human serum. Heat-inactivated human serum was incubated with or without P. gingivalis (10⁸ bacterial cells per ml) for 30 min at 37° C. and C5a generation was determined using a Human C5a ELISA Kit (BD Biosciences). Data are means±SD (n=3) from one of two similar experiments yielding consistent results. **, P<0.01 vs. serum-only control.

FIG. 12 shows the Upregulation of IL-6 production by C5a in P. gingivalis-stimulated macrophages. Mouse peritoneal macrophages were incubated for 5 or 24 hours at 37° C. with P. gingivalis (Pg; MOI=10:1) in the absence or presence of C5a (50 nM) and culture supernatants were assayed for IL-6 by ELISA. Data are means±SD (n=3) from a typical experiment performed three times with consistent results. *, P<0.01 vs. medium control. , P<0.01 in C5a+Pg vs. Pg alone.

Part B: C5a Receptor Impairs IL-12-Dependent Clearance of Porphyromonas gingivalis and is Required for Induction of Periodontal Bone Loss

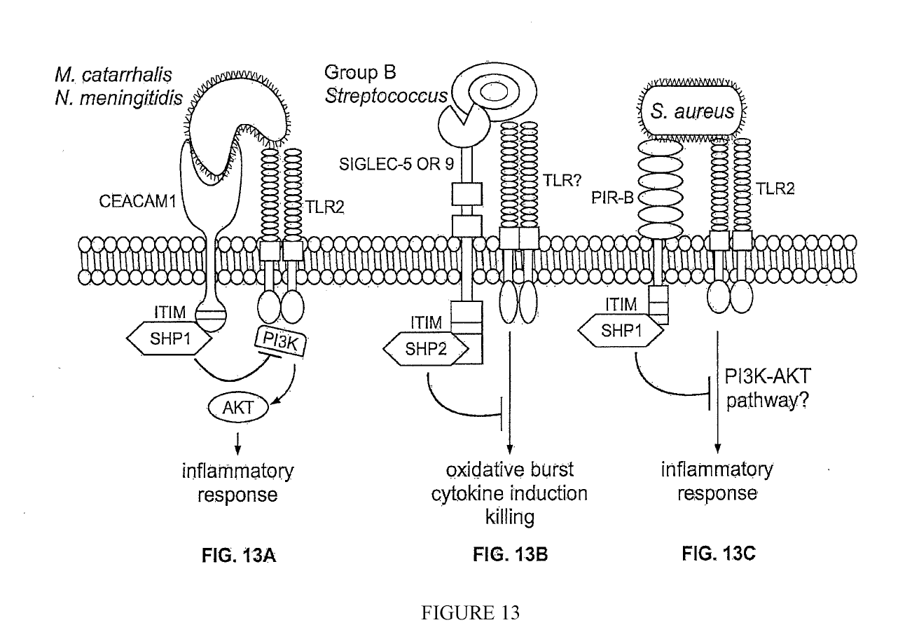

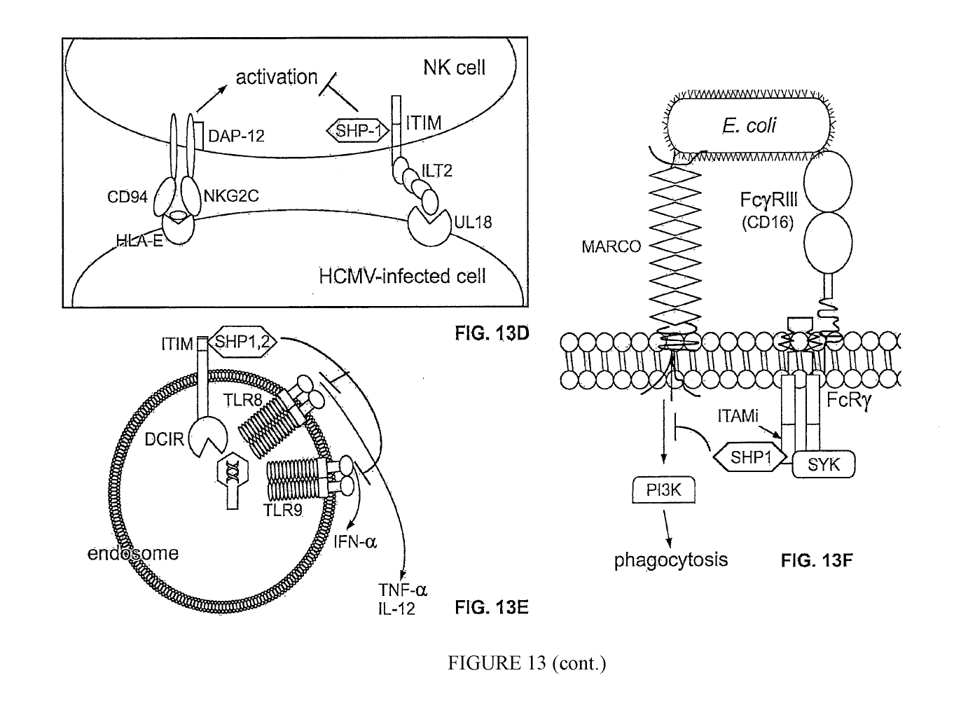

FIG. 13 demonstrates that C5aR signaling inhibits TLR2-dependent IL-12p70 induction in P. gingivalis-activated macrophages. Mouse peritoneal macrophages were primed with IFN-gamma (0.1 μg/ml) and stimulated with medium only (Med), P. gingivalis (MOI 10:1), or E. coli LPS (Ec-LPS; 0.1 μg/ml), as indicated. IFN-gamma priming was performed in those experiments (Panels A-D) investigating IL-12p70 regulation. Wild-type P. gingivalis (Pg) was used in all experiments, but Panel B additionally includes the use of an isogenic mutant (KDP128), which is deficient in all three gingipain genes. In Panels A and B, the macrophages were additionally treated (or not) with C5a (50 nM), in the absence or presence of C5aRA (1 μM). In Panel C, the macrophages were from wild-type or TLR2-deficient (Tlr2^{-/-}) mice. In Panel D, the macrophages were pretreated with U0126 (10 μM) or wortmannin (WTM; 100 nM) for 1 h prior to treatments with C5a, P. gingivalis, or Ec-LPS. In Panel E, the macrophages were stimulated with P. gingivalis as in Panel A, but without IFN-gamma priming, to measure levels of cytokines other than IL-12p70. Culture supernatants were assayed for induction of the indicated cytokines after 24 h of incubation. Data are means±SD (n=3 sets of macrophages) from typical experiments performed three (Panel A) or two (Panels B-E) times. Asterisks show statistically significant (p<0.01) inhibition (Panels A-D; IL-12p70) or enhancement (Panel E; IL-6 and TNF-α) of cytokine production, whereas black circles indicate statistically significant (p<0.01) reversal of these modulatory effects. In Panel B, the upward arrow shows a significant difference (p<0.05) between KDP128 and Pg under no-treatment conditions. In Panel D, inverse triangles show significant (p<0.01) U0126 or WTM effects on P. gingivalis- or LPS-induced IL-12p70.

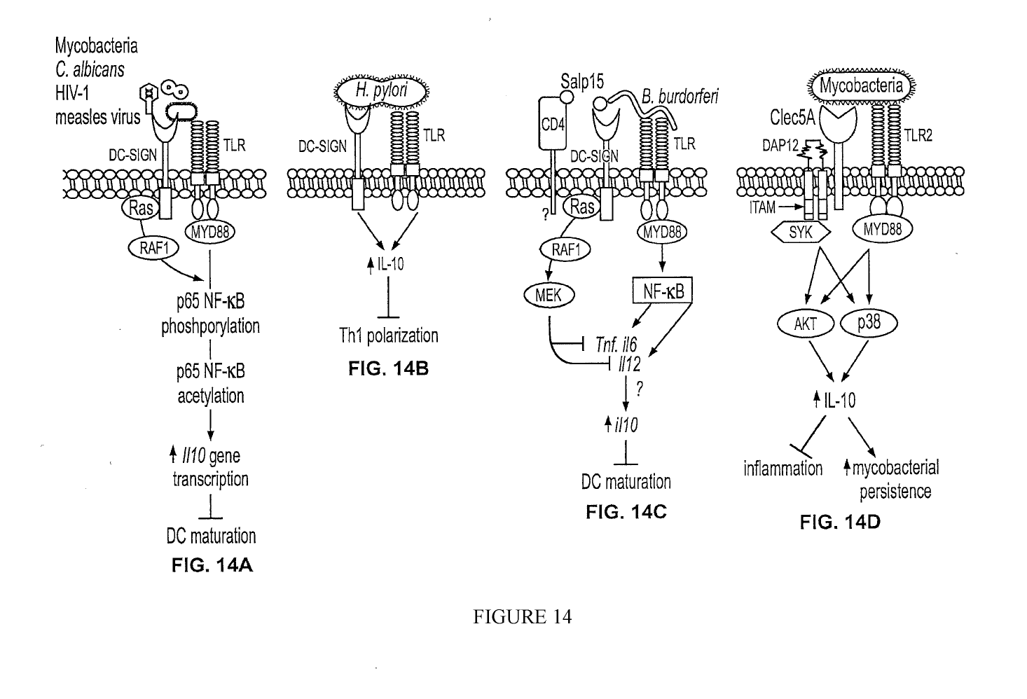

FIG. 14 shows that C5aR signaling regulates P. gingivalis-induced and TLR2-dependent cytokine production in vivo. 10-12 week-old wild-type (WT) mice, which were pretreated or not with C5aRA (i.p.; 25 μg/mouse), as well as mice deficient in C5aR (C5ar^{-/-}) or TLR2 (Tlr2^{-/-}), were i.p. infected with P. gingivalis (5×10⁷ CFU). Peritoneal lavage was performed 5 h post-infection and the peritoneal fluid was used to measure the levels of the indicated cytokines. Mice not infected with P. gingivalis had undetectable levels of the cytokines investigated. Data are means±SD (n=5 mice). *, p<0.01 and **, p<0.01 vs. WT+PBS control.

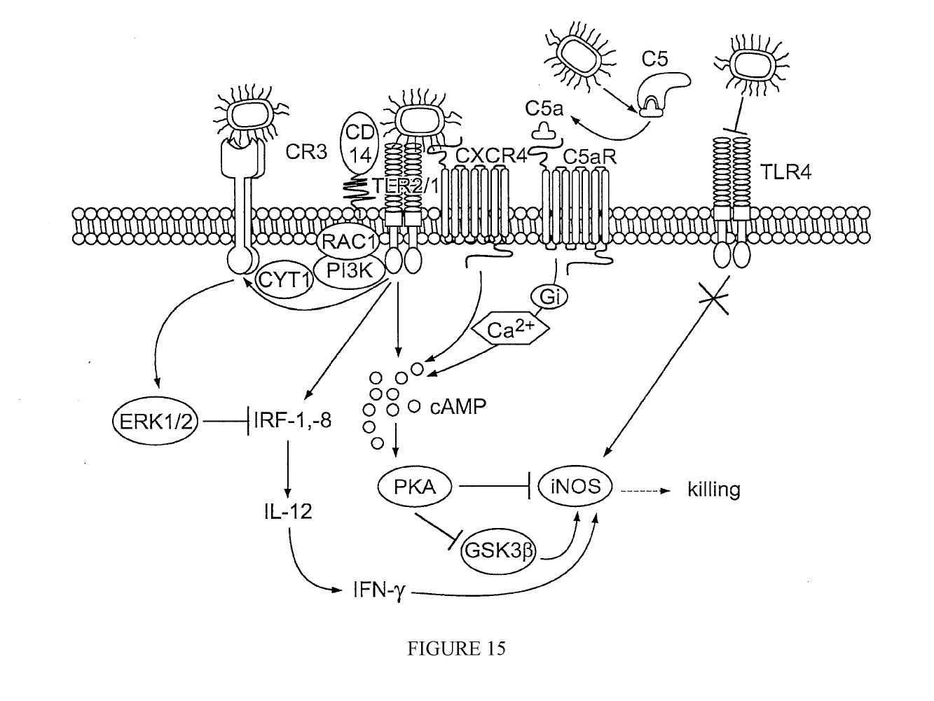

FIG. 15 demonstrates that inhibition of C5aR signaling promotes the in vivo clearance of P. gingivalis by augmenting IL-12. Panel A shows that wild-type (WT) mice were pre-treated (or not) with C5aRA (i.p.; 25 μg/mouse), in the presence or absence of goat polyclonal anti-mouse IL-12 IgG, anti-mouse IL-23p19 IgG, or equal amount of non-immune IgG (i.p.; 0.1 mg/mouse). The mice were then infected i.p. with P. gingivalis (5×10⁷ CFU). Panel B shows a similar experiment in which C5aRA-treated mice were replaced by C5aR-deficient (C5ar^{-/-}) mice. Panel C shows that WT and C5ar^{-/-} mice were infected i.p. with wild-type P. gingivalis or the isogenic KDP128 mutant (both at 5×10⁷ CFU). Peritoneal lavage was performed 24 h post-infection and the peritoneal fluid was used to determine viable P. gingivalis CFU counts. Data are shown for each individual mouse with horizontal lines indicating mean values. *, p<0.01 vs. controls. The inverted triangles indicate significant (p<0.01) reversal of the effects of C5aRA or C5aR deficiency by anti-IL-12. In Panel C, the downward arrow shows significant (p<0.01) difference between KDP128 and the wild-type organism.

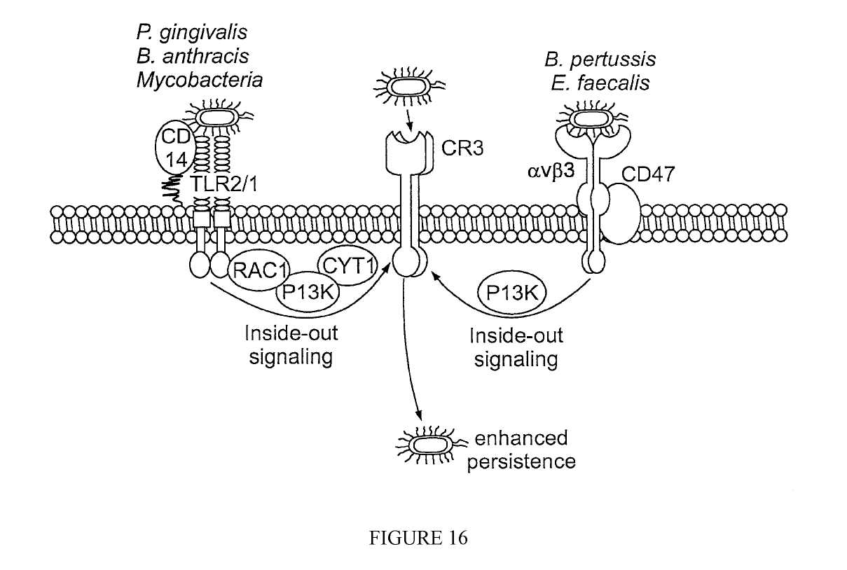

FIG. 16 shows the comparative modulatory effects of C5a and C5a^{desArg on IL-}12p70 production and antimicrobial activities in P. gingivalis-challenged macrophages. Groups of mouse peritoneal macrophages were incubated with P. gingivalis (Pg; MOI=10:1) in the absence or presence of C5a or C5a^{desArg} (at 10 or 50 nM) and assayed for induction of IL-12p70 (after 24 h) (Panel A), generation of cAMP (1 h) (Panel C), NO₂⁻ (24 h) (Panel D), and viable counts (CFU) of internalized bacteria (24 h) (Panel E). In Panel B, the macrophages were pretreated with C5aRA (1 μM), the dual C5aR/C5a-like receptor-2 antagonist A8^{Δ71-73} (1 μM), or the C3aR antagonist SB290157 (5 μM) to determine the receptor by which C5a^{desArg} (50 nM) inhibits IL-12p70 production. Data are means±SD (n=3 sets of macrophages) from one of two independent sets of experiments yielding consistent results. *, p<0.05 and **, p<0.01 compared to no C5a or C5a^{desArg} (0 nM). In Panel B, black circles indicate statistically significant (p<0.01) reversal of the inhibitory effect of C5a^{desArg}. In panels C-E, no significant differences were found between C5a and C5a^{desArg} when tested at 50 nM.

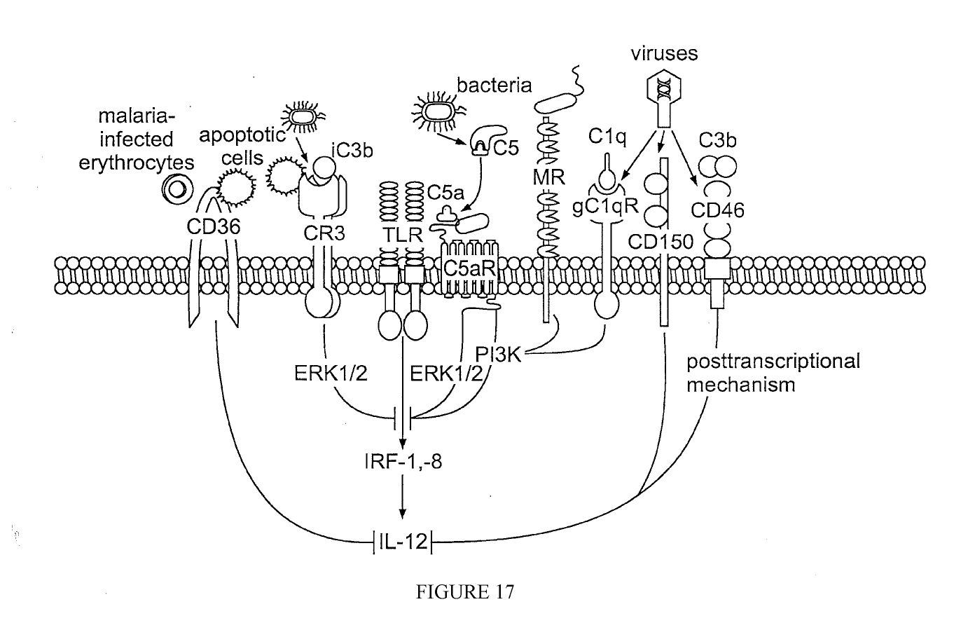

FIG. 17 shows the comparison of C5a and C5a^{desArg} in intracellular Ca²⁺ mobilization. Mouse peritoneal macrophages (Panel A) or neutrophils (Panel B) were loaded with the ratiometric calcium indicator Indo-1 AM and stimulated with C5a or C5a^{desArg} at the indicated concentrations (lower concentrations were used for neutrophils, since they are more sensitive to C5a than macrophages). Ca²⁺ mobilization was measured in a spectrofluorometer and the traces are representative of three experiments.

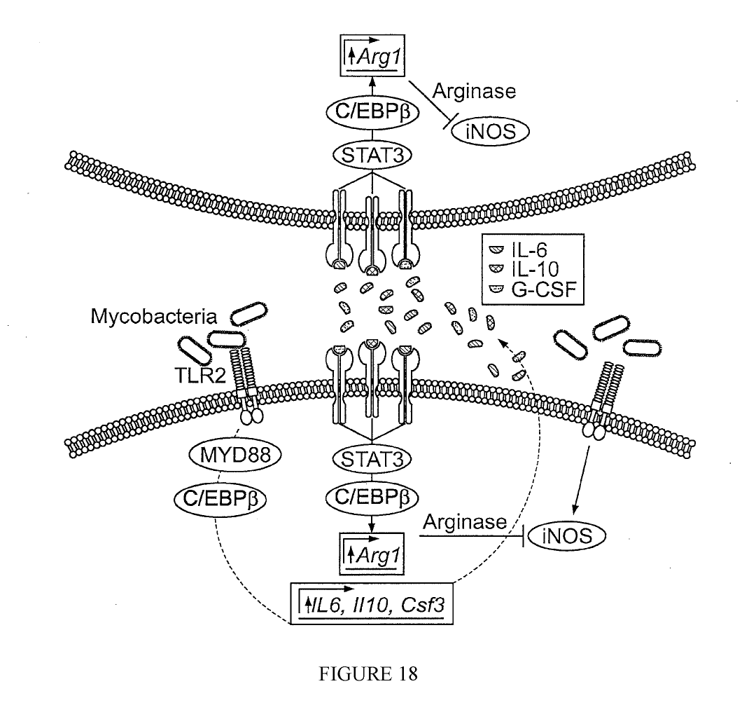

FIG. 18 shows that C5aR and TLR2 deficiencies protect against periodontal bone loss. Mice deficient in C5aR [C5ar^{-/-}] (Panel A, BALB/c; Panel B, C57BL/6) or TLR2 [Tlr2^{-/-}] (Panel C; BALB/c) and appropriate wild-type controls were orally infected (or not) with P. gingivalis and assessed for induction of periodontal bone loss six weeks later. Mice used in these experiments were 10-12 week-old. Panel D shows the induction of naturally occurring periodontal bone loss in 16-month-old wild-type or C5ar^{-/-} BALB/c mice relative to their young counterparts (≦12 weeks of age). Panel E shows representative images of P. gingivalis-induced bone loss under wild-type or C5aR- or TLR2-deficient conditions: P. gingivalis-infected C5ar^{-/-} or Tlr2^{-/-} mice display considerably smaller CEJ-ABC distances (yellow arrows) compared to infected wild-type mice, but quite comparable to those of sham-infected wild-type mice. Data are means±SD (n=5 mice). *, p<0.01 compared to corresponding sham-infected controls (Panels A and B) or young counterparts (Panel C).

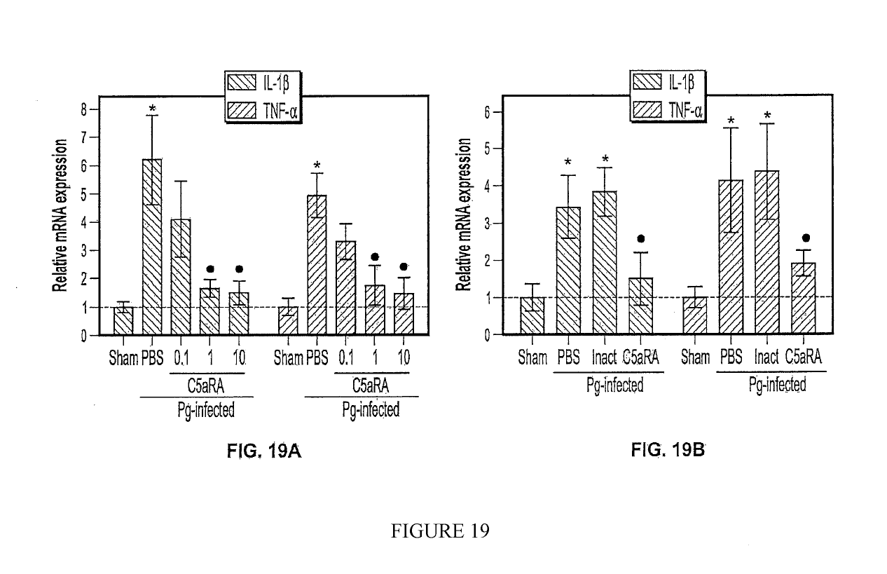

FIG. 19 are graphs showing the preventative (Panel A) and the therapeutic (Panel B) effects of a C5aR antagonist.

Like reference symbols in the various drawings indicate like elements.

### DETAILED DESCRIPTION

Periodontitis is a set of inflammatory diseases affecting the periodontium, i.e., the tissues that surround and support the teeth. Periodontitis involves progressive loss of the alveolar bone around the teeth, and, if left untreated, can lead to the loosening and subsequent loss of teeth. Periodontitis is caused by microorganisms that adhere to and grow on the tooth's surfaces, along with an overly aggressive immune response against these microorganisms. Periodontitis manifests as painful, red, swollen gums, with abundant plaque. Symptoms may include redness or bleeding of gums while brushing teeth, using dental floss, or biting into hard food (e.g. apples); recurrent swelling of the gum; halitosis and a persistent metallic taste in the mouth; gingival recession resulting in apparent lengthening of teeth; deep pockets between the teeth and the gums (pockets are sites where the attachment has been gradually destroyed by collagenases); and loose teeth.

In 1999, a classification system was developed for periodontal diseases and conditions, which listed seven major categories of periodontal diseases, of which the last six are termed “destructive periodontal disease” because they are essentially irreversible. In addition, terminology expressing both the extent and severity of periodontal diseases are appended to the classes to further denote the specific diagnosis. The extent of disease refers to the proportion of the dentition affected by the disease in terms of percentage of sites. Sites are defined as the positions at which probing measurements are taken around each tooth and, generally, six probing sites around each tooth are recorded to make a determination of the extent of periodontal disease. Typically, if up to 30% of sites in the mouth are affected, the manifestation is classification as localized; if more than 30% of sites in the mouth are affected, the term generalized is used. The severity of disease refers to the amount of periodontal ligament fibers that have been lost, termed clinical attachment loss, and is defined by the American Academy of Periodontology as mild (1-2 mm of attachment loss), moderate (3-4 mm of attachment loss), or severe (≧5 mm of attachment loss).

Periodontitis also has been shown to have effects outside of the mouth. For example, periodontitis has been linked to increased inflammation as indicated by increased levels of C-reactive protein and Interleukin-6. In addition, periodontitis has been shown to increase the risk for a number of other diseases, including but not limited to, stroke, myocardial infarction, atherosclerosis, diabetes, and pre-term labor.

The primary pathogen involved in periodontitis is Porphyromonas gingivalis, a gram-negative anaerobic bacterium. P. gingivalis inhibits the complement cascade and, surprisingly, induces a subversive crosstalk between the complement C5a receptor (C5aR) and TLR2 that impairs nitric oxide-dependent intracellular killing in macrophages. Interestingly, P. gingivalis can control both receptors: it can directly engage TLR2 through cell-surface ligands, and it can activate C5aR (CD88) through conversion of C5 to C5a using its own cysteine proteinases (gingipains). Indeed, P. gingivalis does not have to rely on an immunological response by the host to generate C5a. However, since C5a is a powerful chemoattractant and activator of phagocytes, it would seem counterproductive for a pathogen to actively contribute to C5a generation.

As described herein, P. gingivalis paradoxically employs the proinflammatory C5a for targeted immune suppression of macrophages through a novel crosstalk mechanism between the C5a receptor (C5aR) and TLR2, the predominant TLR utilized by P. gingivalis. This is the first report of a pathogen being capable of proactively instigating and exploiting crosstalk signaling between complement and TLRs, rather than undermining one or the other system independently as previously shown for a number of other microbes. In addition, P. gingivalis is the first pathogen shown to exploit complement and TLRs to cause cAMP-dependent immune subversion. This sophisticated subversive crosstalk instigated by P. gingivalis serves in lieu of “built-in” adenylate cyclase which is not expressed by this bacterium, in contrast to Bordetella pertussis, for example, which disables human or mouse phagocytes by means of its own adenylate cyclase. Therefore, this work constitutes the first report of pathogen-induced complement-TLR crosstalk for synergistic cAMP induction to disable macrophages.

Methods of Treating or Preventing Periodontitis or Diseases Associated with Periodontitis

The mechanisms used by P. gingivalis to overcome and thwart the host's immune response as described herein can be used against the pathogen in methods of treating or preventing periodontitis or diseases associated with periodontitis. For example, blocking C5aR or TLR2 effectively deprives P. gingivalis of crucial survival tactics. Thus, methods that inhibit or block C5a receptor expression, activity or activation or TLR2 expression or activity can be used to reduce the amount of P. gingivalis in an individual, thereby protecting the individual from periodontitis and associated systemic diseases like atherosclerosis. In addition, methods that inhibit or block the crosstalk between C5aR and TLR2, or that inhibit the immunosuppressive signaling that occurs in the presence of the C5aR and TLR2, also can be used to reduce the amount of P. gingivalis in an individual, thereby protecting the individual from periodontitis and associated systemic diseases.

Such methods (e.g., methods of inhibiting or blocking C5aR expression, activity or activation; methods of inhibiting or blocking TLR2 expression or activity; or methods of inhibiting or blocking the crosstalk between C5aR and TLR2 or the immunosuppressive signaling that occurs as a result of such crosstalk) typically include administering a compound to the individual that inhibits or blocks C5a receptor expression, activity or activation; a compound that inhibits or blocks TLR2 expression or activity; or a compound that inhibits or blocks the crosstalk between C5aR and TLR2 or the immunosuppressive signaling that occurs as a result of such crosstalk.

By way of example, there are a number of compounds that are known to inhibit or block C5a receptor expression, activity, or activation (e.g., C5a receptor antagonists). For example, acetylated phenylalanine-(ornithine-proline-(D)cyclohexylalanine-tryptophan-arginine) is a small molecule antagonist of the human C5a receptor (see, for example, Woodruff et al., 2003, J. Immunol., 171:5514-20), as is W-54011 (see, for example, Sumichika et al., 2002, J. Biol. Chem., 277:49403-7), ADC-1004 (see, for example, van der Pals et al., 2010, BMC Cardiovasc. Disord., 10:45), CGS 32359 (see, for example, Riley et al., 2000, J. Thorac. Cardiovasc. Surg., 120:350-8), NDT9520492 (see, for example, Waters et al., 2005, J. Biol. Chem., 280:40617-23), NGD 2000-1 (see, for example, Lee et al., 2008, Immunol. Cell Biol., 86:153-60), CP-447,697 (Blagg et al., 2008, Bioorg. Med. Chem. Lett., 18:5601-4), and NDT 9513727 (Brodbeck et al., 2008, J. Pharmacol. Exp. Ther., 327:898-909). In addition, a number of peptidomimetics have been identified as useful C5aR antagonists, including, without limitation, C089 (see, for example, Konteatis et al., 1994, J. Immunol., 153:4200-5), PMX-53 (see, for example, Finch et al., 1999, J. Med. Chem., 42:1965-74), PMX-205 (see, for example, March et al., 2004, Mol. PharmacoL, 65:868-79), and JPE-1375 (see, for example, Schnatbaum et al., 2006, Bioorg. Med. Chem. Letters, 16:5088-92). In addition, Strachan et al. (2000, J, Immunol., 164:6560-5), Heller et al. (1999, J. Immunol., 163:985-94), Pellas et al. (1999, Current Pharm. Design, 5:737-55), and U.S. Pat. No. 7,727,960 to Hummel et al. disclose additional examples of C5a receptor antagonists. See, also, Qu et al., 2009, Mol. Immunol., 47:185-95.

An antibody against the C5a receptor also can be used to inhibit or block C5a receptor expression, activity, or activation. Antibodies against C5aR are known (see, for example, Morgan et al., 1993, J. Immunol., 151:377-88; Guo et al., 2006, Recent Pat. Antiinfect. Drug Discov., 1:57-65; and Zhang et al., 2007, Biochem. Biophys. Res. Commun., 357:446-52), and are commercially available from Pierce Antibodies (Rockford, Ill.), CedarLane Laboratories Ltd. (Hornby, Ontario), and GenWay (San Diego, Calif.). G2 Therapies also has a therapeutic antibody in preclinical trials, referred to as Neutrazumab, directed toward the C5aR. In addition, RNA interference (“RNAi”) can be used to specifically target the nucleic acid encoding the C5a receptor. RNAi is a process that is used to induce specific post-translational gene silencing. RNAi involves introduction of RNA with partial or fully double-stranded character into the cell or into the extracellular environment. The portion of the target gene used to make RNAi can encompass exons but also can include untranslated regions (UTRs) as well as introns. See, for example, Kim et al., 2008, Biotechniques, 44:613-6 as well as Lares et al., 2010, Trends Biotechnol., 28:570-9; and Pfeifer et al., 2010, Pharmacol. Ther., 126:217-27. See, also, Ricklin & Lambris, 2007, Nature Biotechnol., 25:1265-75.

In certain embodiments, one or more inhibitors of complement can be administered to an individual and used to prevent or treat periodontitis (or diseases associated with periodontitis) via the role of complement, as described herein, in the formation of periodontitis and, specifically, in the establishment of P. gingivalis. Representative complement inhibitors include, without limitation, sCR1, C1 Inhibitor (C1inh), Membrane Cofactor Protein (MCP), Decay Accelerating Factor (DAF), MCP-DAF fusion protein (CAB-2), C4 bp, Factor H, Factor I, Carboxypeptidase N, vitronectin (S Protein), clusterin, CD59, compstatin and its functional analogs, C1q inhibitors or anti-C1q antibodies, C1 inhibitors or anti-C1 antibodies, C1r inhibitors or anti-C1r antibodies, C1s inhibitors or anti-C1s antibodies, MSP inhibitors or anti-MASP antibodies, MBL inhibitors or anti-MBL antibodies, C2 inhibitors or anti-C2 antibodies, C4 inhibitors or anti-C4 antibodies, C4a inhibitors or anti-C4a antibodies, C5 inhibitors or anti-C5 antibodies, C5a inhibitors or anti-05a antibodies, C5aR inhibitors or anti-C5aR antibodies, C5b inhibitors or anti-C5b antibodies, C3 inhibitors or anti-C3 antibodies, C3a inhibitors or anti-C3a antibodies, C3aR inhibitors or anti-C3aR antibodies, C6 inhibitors or anti-C6 antibodies, C7 inhibitors or anti-C7 antibodies, C8 inhibitors or anti-C8 antibodies, C9 inhibitors or anti-C9 antibodies, properdin inhibitors or anti-properdin antibodies, Factor B inhibitors or anti-Factor B antibodies, or Factor D inhibitors or anti-Factor D antibodies.

Compounds that inhibit or block C5aR or TLR2 expression, activity, or crosstalk can be administered to an individual via any number of routes, which typically depends on the particular compound and its features. Compounds can be incorporated into pharmaceutical compositions suitable for administration to an individual. Such compositions typically include, at least, the compound and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Additional or secondary active compounds also can be incorporated into the compositions described herein.

A pharmaceutical composition as described herein is formulated to be compatible with its intended route of administration. Examples of routes of administration include parenteral, e.g., intravenous, intradermal, subcutaneous, oral (e.g., ingestion or inhalation), transdermal (topical), transmucosal, and rectal administration. In addition, local administration into the periodontal pocket (e.g., via direct injection, or via, for example, a Perio Chip) also is a route of administration that may be employed in the methods described herein. Solutions or suspensions used for parenteral, intradermal, or subcutaneous application can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and/or antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and/or by the use of surfactants. In many cases, it will be preferable to include isotonic agents, for example, sugars, polyalcohols such as mannitol or sorbitol, and sodium chloride in the composition. Prolonged administration of an injectable composition can be brought about by including an agent that delays absorption. Such agents include, for example, aluminum monostearate and gelatin. A parenteral preparation can be enclosed in ampoules, disposable syringes or multiple dose vials made of glass or plastic.

Oral compositions generally include an inert diluent or an edible carrier. Oral compositions can be liquid, or can be enclosed in gelatin capsules or compressed into tablets. Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. Transmucosal administration can be accomplished through the use of nasal sprays or suppositories. For transdermal administration, the active compounds typically are formulated into ointments, salves, gels, or creams as generally known in the art.

It is especially advantageous to formulate oral or parenteral compositions in dosage unit form for ease of administration and uniformity of dosage. Dosage unit form as used herein refers to physically discrete units suited as unitary dosages for an individual to receive; each unit containing a predetermined quantity of active compound calculated to produce the desired therapeutic effect in association with the required pharmaceutical carrier. The dosage units themselves are dependent upon the amount of compound to be delivered. The amount of a compound necessary to inhibit or block C5a receptor expression, activity or activation, or inhibit or block the crosstalk between C5aR and TLR2 or the immunosuppressive signaling that occurs as a result of such crosstalk can be formulated in a single dose, or can be formulated in multiple dosage units. Treatment of an individual with a compound that inhibits or blocks C5a receptor expression, activity or activation, or a compound that inhibits or blocks the crosstalk between C5aR and TLR2 or inhibits the immunosuppressive signaling that occurs as a result of such crosstalk, may require a one-time dose, or may require repeated or multiple doses.

Screening for Compounds that can be Used to Treat or Prevent Periodontitis or Diseases Associated with Periodontitis

The results described herein regarding the role of C5aR, TLR2, and the crosstalk between C5aR and TLR2 that is induced by P. gingivalis also can be used to screen for therapeutic compounds (i.e., compounds that inhibit the expression, activity, or activation of C5aR, the expression or activity of TLR2, or the crosstalk between C5aR and TLR2).

For example, a nucleic acid molecule can be produced that includes a promoter operably linked to nucleic acid encoding a C5aR polypeptide or a TLR2 polypeptide. Promoters that drive expression of a DNA sequence are well known in the art. Promoters suitable for expressing a nucleic acid encoding C5aR or TLR2 would be known to those skilled in the art and include, for example, constitutive or inducible promoters. Many constitutive and inducible promoters are known in the art. As used herein, “operably linked” means that a promoter and/or other regulatory element(s) are positioned in a vector relative to a nucleic acid encoding C5aR or TLR2 in such a way as to direct or regulate expression of the nucleic acid. Such a nucleic acid molecule can be introduced into host cells (e.g., E. coli, yeast) using routine methods (e.g., electroporation, lipid-based delivery systems, nanoparticle delivery systems, and viral-based delivery systems), and the host cells can be contacted with a test compound. A vector as described herein also may include sequences such as those encoding a selectable marker (e.g., an antibiotic resistance gene).

Methods of evaluating whether or not a test compound inhibits the expression of C5aR or TLR2 are well known in the art. For example, RT-PCR or Northern blotting methods can be used to determine the amount of C5aR or TLR2 mRNA in the presence and absence of the test compound. In addition, methods that can be used to evaluate whether or not a test compound inhibits the activity or the activation of C5 aR or TLR2 are well known in the art. For example, methods of determining whether or not a test compound inhibits the activity of G protein-coupled receptors are known in the art as are methods of evaluating whether or not a test compound inhibits the activation of C5aR. See, for example, Hipser et al., 2010, Mt. Sinai J. Med., 77:374-80; Scott et al., 2010, Drug Discov. Today, 15:704-16; Bortolato et al., 2009, and Curr. Pharm., Des., 15:4017-25.

In addition, the results described herein regarding the crosstalk between C5aR and TLR2 induced by P. gingivalis also can be used to screen for compounds that inhibit that crosstalk or that inhibit the immunosuppressive signaling that occurs due to that crosstalk. In certain embodiments, a recombinant cell can be produced having all of the necessary components to evaluate the crosstalk between C5aR and TLR2 in the presence of a test compound. For example, a recombinant host cell can be generated that includes exogenous nucleic acids encoding either or both the C5aR polypeptide and the TLR2 polypeptide. In certain instances, one or more exogenous nucleic acids encoding downstream product(s) (e.g., one or more cytokines such as IL-6 or TNF-alpha) also are introduced into the recombinant host cell; in other instances, the host cell naturally produces such downstream products (e.g., via endogenous nucleic acids). For example, mammalian host cells would naturally contain TLR2, complement factors including C5aR, and the downstream products resulting from of affected by the crosstalk.

Methods of making recombinant host cells (e.g., recombinant mammalian host cells) are discussed herein and are well known in the art. In addition, the crosstalk instigated by P. gingivalis is described herein, and representative methods of evaluating the crosstalk and the downstream effects resulting from that crosstalk are shown in the Examples.

Virtually any type of compound can be used as a test compound in the screening methods described herein. Test compounds can include, for example and without limitation, nucleic acids, peptides, proteins, non-peptide compounds, synthetic compounds, peptidomimetics, antibodies, small molecules, fermentation products, or extracts (e.g., cell extracts, plant extracts, or animal tissue extracts).

In accordance with the present disclosure, there may be employed conventional molecular biology, microbiology, biochemical, and recombinant DNA techniques within the skill of the art. Such techniques are explained fully in the literature. The discovery will be further described in the following examples, which do not limit the scope of the methods and compositions of matter described in the claims.

### EXAMPLES

### Part A: Microbial Hijacking of Complement-Toll-Like Receptor Crosstalk

### Example 1—Reagents

SQ22536, H89, SB216367, 8-Br-cAMP, AMD3100, forskolin, L-NAME (N(G)-nitro-L-arginine methyl ester), D-NAME (N(G)-nitro-D-arginine methyl ester), and EGTA were purchased from Sigma-Aldrich Chemical Co. Chelelythrin, PKI 6-22, KT5823, and thapsigargin were obtained from Calbiochem. PD98059 was from Cell Signaling Technology. Mouse-specific monoclonal antibodies to TLR2 [clone 6C2] was from e-Bioscience, TLR5 [85B152.5] from Abeam, and C5aR (20/70) from Cedarlane Laboratories or Hycult. Mouse IFN-γ was from R&D Systems. Mouse C5a was purchased from Cell Sciences or R&D Systems, and C3a from R&D Systems. The cyclic hexapeptide AcF(OP(D)ChaWR) (acetylated phenylalanine-(ornithine-proline-(D)cyclohexylalanine-tryptophan-arginine)), a specific and potent C5a receptor (CD88) antagonist, was synthesized as previously described (Finch et al., 1999, J. Med. Chem., 42:1965-74; Markiewski et al., 2008, Nat. Immunol., 9:1225-25). C5a and C3a were used at concentrations up to 100 nM and 200 nM, respectively, which are widely used in in vitro experiments. Moreover, these concentrations are consistent with observations that, under inflammatory conditions, C5a and C3a may reach serum levels as high as 100 nM and 400 nM, respectively, although even higher levels may be generated at local sites of inflammation. All reagents were used at optimal concentrations determined in preliminary or published studies (Hajishengallis et al., 2008, PNAS USA, 105:13532-7; Markiewski et al., 2008, Nat. Immunol., 9:1225-35; Liang et al., 2007, J. Immunol., 178:4811-9). When appropriate, dimethyl sulfoxide (DMSO) was included in medium controls at a final concentration of ≦0.2%.

### Example 2—Bacteria and Mammalian Cells

P. gingivalis ATCC 33277 was grown anaerobically from frozen stocks on modified Gifu anaerobic medium (GAM)-based blood agar plates for 5-6 days at 37° C., followed by anaerobic subculturing for 18-24 hours at 37° C. in modified GAM broth (Nissui Pharmaceutical). Thioglycollate-elicited macrophages were isolated from the peritoneal cavity of wild-type or mice deficient in TLR2, TLR4, C3aR, or C5aR (The Jackson Laboratory) (Zhang et al., 2007, Blood, 110:228-36; Gajishengallis et al., 2006, Cell. Microbiol., 8:1557-70), in compliance with established federal guidelines and institutional policies. The macrophages were cultured at 37° C. and 5% CO₂ in RPMI 1640 (InVitrogen) supplemented with 10% heat-inactivated FBS, 2 mM L-glutamine, 100 units/ml penicillin G, 100 μg/ml streptomycin, and 0.05 mM 2-ME. None of the experimental treatments, including treatments with C5a up to 100 nM, affected cell viability (monitored by the CellTiter-Blue™ assay; Promega) compared to medium-only treatments.

### Example 3—Intracellular Survival Assay

The viability of phagocytosed P. gingivalis was monitored by an antibiotic protection-based intracellular survival assay, as previously described (Wang et al., 2007, J. Immunol., 179:2349-58). Briefly, mouse peritoneal macrophages were allowed to phagocytose P. gingivalis (MOI=10:1; 5×10⁶ bacteria and 5×10⁵ cells) for 30 min at 37° C. This was followed by washing to remove extracellular nonadherent bacteria and 1-hour treatment with antibiotics (300 μg/ml gentamicin and 200 μg/ml metronidazole) to eliminate residual or extracellular adherent bacteria. The macrophages were subsequently cultured overnight (for a total of 24 hours) or for 48 hours. Immediately after, the macrophages were washed and lysed in sterile distilled water, and viable counts of internalized P. gingivalis were determined by plating serial dilutions of macrophage lysates on blood agar plates subjected to anaerobic culture.

### Example 4—Cell Signaling and Activation Assays

Induction of nitric oxide production was assessed by measuring the amount of NO₂⁻ (stable metabolite of nitric oxide) in stimulated culture supernatants using a Griess reaction-based assay kit (R&D Systems), as previously performed (Hajishengallis et al., 2008, PNAS USA, 105:13532-7). Levels of cAMP in activated cell extracts were measured using a cAMP enzyme immunoassay kit (Cayman Chemical) (Liang et al., 2007, J. Iminunol., 178:4811-9). PKA activity in lysates of activated cells was determined using the ProFluor™ assay, according to the instructions of the manufacturer (Promega) (Hajishengallis et al., 2008, PNAS USA, 105:13532-7). Phosphorylation of GSK3β on Ser9 and total GSK3β were monitored using FACE™ GSK3β ELISA kits (Active Motif).

### Example 5—In Vivo Infection

Upon i.p. infection of mice with P. gingivalis (5×10⁷ CFU), peritoneal lavage was performed 24 hours post-infection and the peritoneal fluid was used to enumerate recovered CFU (following anaerobic growth on blood agar plates) and measure production of NO₂⁻ (as described in Hajishengallis et al., 2008, PNAS USA, 105:13532-7). All animal procedures were approved by the Institutional Animal Care and Use Committee and performed in compliance with established federal and state policies.

### Example 6—Quantitative Real-Time PCR

Gene expression in resting or activated mouse macrophages was quantified using quantitative real-time PCR. Briefly, RNA was extracted from cell lysates using the PerfectPure RNA cell kit (5 Prime, Fisher) and quantified by spectrometry at 260 and 280 nm. The RNA was reverse-transcribed using the High-Capacity cDNA Archive kit (Applied Biosystems) and quantitative real-time PCR with cDNA was performed using the ABI 7500 Fast System, according to the manufacturer's protocol (Applied Biosystems). TaqMan probes, sense primers, and antisense primers for expression of a house-keeping gene (GAPDH) or iNOS (or the genes shown in FIG. 7) were purchased from Applied Biosystems.

### Example 7—Confocal Microscopy

To examine co-localization of P. gingivalis with C5aR and TLR2, mouse macrophages were grown on chamber slides and exposed to FITC-labeled P. gingivalis for 10 min. The cells were then fixed, permeabilized, stained with Texas Red-labeled anti-05aR plus allophycocyanin-labeled anti-TLR2, and mounted with coverslips for imaging on an Olympus FV500 confocal microscope.

### Example 8—Fluorescence Resonance Energy Transfer (FRET)

Upon stimulation for 10 min at 37° C. with P. gingivalis, mouse macrophages were labeled with a mixture of Cy3-conjugated (donor) and Cy5-conjugated (acceptor) antibodies, as indicated in FIG. 4I. In additional experiments shown in FIG. 4I, FITC-labeled P. gingivalis was used as donor and TRITC-labeled receptors served as acceptors. The cells were washed and fixed, and energy transfer between various donor-acceptor pairs was calculated from the increase in donor fluorescence after acceptor photobleaching (REF 9, 14). The maximum (max) and minimum (min) energy transfer efficiencies in the experimental system were determined in control experiments as the energy transfer between two different epitopes on the same molecule or between molecules that do not engage in heterotypic associations, and their values are denoted by dashed lines in FIG. 4I. The conjugation of antibodies to Cy3 or Cy5 was performed using kits from Amersham Biosciences.

### Example 9—Statistical Analysis

Data were evaluated by analysis of variance and the Dunnett multiple-comparison test using the InStat program (GraphPad Software, San Diego, Calif.). Where appropriate (comparison of two groups only), two-tailed t tests were performed. P<0.05 was taken as the level of significance. All experiments were performed at least twice for verification.

### Example 10—C5a and Subversion of Macrophage Function

Whether C5a influences the macrophage intracellular killing of P. gingivalis was examined. Strikingly, the ability of this pathogen to survive intracellularly in mouse macrophages was significantly promoted by C5a, but not by the related anaphylatoxin C3a (FIGS. 1A and 1B). This unexpected pro-microbial effect of C5a was enhanced with increasing concentrations of C5a. (FIG. 5A) and was also observed in interferon (IFN)-gamma-primed macrophages (FIGS. 1C and 1D). The elevated viable cell counts of P. gingivalis in C5a-treated macrophages could not be attributed to possible differences in the initial bacterial loads, since P. gingivalis phagocytosis was not significantly affected by the absence or presence of C5a or C3a (FIG. 6A). Consistent with this, the expression of macrophage receptors, which coordinately mediate P. gingivalis uptake, such as CD14, TLR2, and CD11b/CD18, was essentially unaffected by C5a (FIGS. 6B and 6C).

The mechanism(s) underlying C5a-mediated inhibition of the macrophage intracellular killing capacity was investigated next. In this regard, it was hypothesized that the combined action of C5a and P. gingivalis on macrophages may induce immunosuppressive signaling. Real-time quantitative PCR was used to determine whether C5a up-regulates the expression of negative regulators of TLR signaling in P. gingivalis-stimulated macrophages. Although the bacterium alone up-regulated the expression of some of the investigated regulators, including the suppressor of cytokine signaling-1, the interleukin-1 receptor-associated kinase M, and the ubiquitin-editing enzyme A20, no synergistic or additive effects were seen in the concomitant presence of P. gingivalis and C5a (FIG. 7). Therefore, these regulatory molecules are not likely involved in C5a-mediated suppression of macrophage killing of P. gingivalis. Moreover, although induction of cAMP can induce immunosuppressive signaling, C5a by itself failed to induce a cAMP response in macrophages (FIG. 1E). Strikingly, however, C5a synergized with P. gingivalis resulting in >3-fold elevation of the intracellular cAMP levels relative to P. gingivalis stimulation alone (FIG. 1E). The synergy was observed as early as 10 min after cell stimulation, peaked at 1 hour, but significantly elevated cAMP levels were sustained for at least 24 hours (FIG. 1E). This up-regulatory effect of C5a was dose-dependent (FIG. 5B) and was totally abrogated by a C5aR antagonist (C5aRA), the cyclic hexapeptide AcF(OP(D)ChaWR) (FIG. 1F), indicating that C5a acted through the classic C5aR (CD88), rather than the alternative C5a-like receptor 2.

Given that P. gingivalis is exquisitely resistant to killing by the oxidative burst, whether C5a interferes with induction of nitric oxide was investigated as a possible mechanism for its promicrobial effect. The underlying rationale was that P. gingivalis is sensitive to nitric oxide-mediated killing. Indeed, C5a significantly inhibited, via a C5aR-dependent mechanism, the production of nitric oxide in P. gingivalis-stimulated macrophages, even in cells primed with IFN-gamma (FIG. 1G). The C5aR specificity of the C5a-driven augmentation of cAMP and suppression of nitric oxide in P. gingivalis-challenged macrophages was confirmed by lack of these effects in C5aR-deficient (C5ar^{-/-}) macrophages (FIGS. 1H and 1I, respectively). The inhibitory effect of C5a on nitric oxide was dose-dependent (FIGS. 8A and 8B), although it progressively declined with increasing delay of C5a addition to the P. gingivalis-infected macrophages (FIGS. 8C and 8D), suggesting a requirement for an early crosstalk between C5a- and P. gingivalis-induced signaling. On the other hand, when C5a was added together with P. gingivalis, the inhibitory C5a effect was maintained for at least 48 hours (FIGS. 8E and 8F). The FIG. 1 findings suggest that C5aR activation by C5a results in suppression of P. gingivalis intracellular killing associated with elevation of cAMP and reduction of nitric oxide. Cause-and-effect relationships were established in subsequent experiments described in more detail below.

### Example 11—C5a Immunosubversive Effects are Strictly Dependent on cAMP-PKA Signaling

Whether the C5a-mediated inhibition of nitric oxide production depends upon the ability of C5a to stimulate synergistic elevation of cAMP was investigated. Indeed, the inhibitory C5a effect on nitric oxide was reversed in macrophages pretreated with inhibitors of cAMP synthesis (SQ22536) or of PKA (H89 and PKI 6-22) but not with inhibitors of irrelevant kinases (chelerythrin or KT5823) (FIG. 2A), indicating that the C5a effect is mediated by cAMP-dependent PKA signaling. Importantly, the up-regulation of nitric oxide levels by inhibitors of cAMP or of PKA was linked to significantly reduced intracellular survival of P. gingivalis in those same cells (FIG. 2B). Moreover, macrophage pretreatment with C5aRA counteracted the protective effect of C5a on P. gingivalis intracellular viability, whereas L-NAME (nitric oxide synthesis inhibitor) mimicked C5a and overrode the C5aRA effect (FIG. 2C). In contrast, D-NAME, an inactive enantiomer control, had no effect in that regard (FIG. 2C). Interestingly, the ability of inhibitors of cAMP or of PKA to reverse the immunosuppressive C5a effect progressively declined with increasing delay of their addition to the culture system (FIG. 2D). Therefore, P. gingivalis needs to immediately activate cAMP-dependent PKA signaling to suppress the macrophage killing capacity, consistent with the requirement for early availability of C5a in order to disable P. gingivalis-challenged macrophages (FIGS. 8C and 8D).

### Example 12—In Vivo Exploitation of C5aR Signaling for Inhibition of Nitric Oxide and Promotion of Microbial Survival

To determine if C5aR signaling promotes P. gingivalis virulence also in vivo, the pathogen's ability to survive in mice after intraperitoneal infection was investigated, in the absence or presence of C5aRA. At 24 hours post-infection, the peritoneal lavage fluid from C5aRA-treated mice contained significantly lower P. gingivalis CFU compared to control mice (>95% reduction; FIG. 3A). Consistent with this, C5ar^{-/-} mice were superior to wild-type controls in controlling the P. gingivalis infection (FIG. 3A). The wild-type control mice were additionally found to be bacteremic for P. gingivalis (4 out of 5 mice in this group had positive blood cultures 24 hours post-infection), whereas no bacteremia could be detected in C5ar^{-/-} or C5aRA-treated wild-type mice, further indicating that C5aR signaling promotes P. gingivalis virulence. Additional support that the reduced peritoneal bacterial burden in the absence of C5aR signaling reflects increased P. gingivalis killing (rather than P. gingivalis escaping and taking up residence in internal organs) was obtained by lack of P. gingivalis CFU detection in homogenates of several organs examined (spleen, kidney, liver, and lungs) from either C5ar^{-/-} or wild-type mice. The ability of C5aRA-treated mice for enhanced clearance of P. gingivalis correlated with elevated nitric oxide production (relative to control mice), whereas L-NAME counteracted both effects (FIGS. 3B and 3C). Therefore, as shown in vitro, the in vivo exploitation of C5aR signaling by P. gingivalis for enhanced survival involves a nitric oxide-dependent mechanism.

### Example 13—Synergistic Activation of the cAMP-PKA Pathway Requires C5aR-TLR2 Crosstalk

A systematic analysis of crosstalk in intracellular signaling pathways has revealed that receptor-mediated elevation of intracellular Ca²⁺ may potentiate cAMP induction by appropriate stimuli. If the synergistic effect of C5a on cAMP induction (FIG. 1E) depends upon its Ca²⁺-mobilizing activity, then this synergy should be inhibited by thapsigargin, an inhibitor of the endoplasmic reticulum Ca²⁺-ATPase, which blocks the C5a-induced intracellular Ca²⁺ response. Indeed, macrophage pre-treatment with thapsigargin abrogated the synergistic C5a effect on P. gingivalis-induced cAMP, whereas EGTA, which chelates extracellular Ca²⁺, had a relatively minimal and statistically insignificant effect (FIG. 4A). Significant reversal of the C5a effect on cAMP induction was also seen in cells pre-treated with pertussis toxin (FIG. 4A), suggesting Gαᵢ-coupled C5aR signaling.

In the absence of C5a, the ability of P. gingivalis to induce cAMP depends on its interaction with the CXC-chemokine receptor 4 (CXCR4). Thus, it was initially speculated that the synergistic C5a effect on cAMP induction could involve a crosstalk between C5aR and CXCR4. Although CXCR4 blockade by AMD3100 (at 1 μg/ml, which completely inhibits the CXCR4-P. gingivalis interaction) modestly attenuated the synergistic C5a effect on cAMP production, the synergism was still profoundly manifested (>6-fold difference between AMD+C5a+Pg vs. AMD+Pg; FIG. 4B). Moreover, P. gingivalis failed to elevate intracellular cAMP in CXCR4-transfected CHO-K1 cells, although it induced cAMP production in cells cotransfected with CXCR4 and TLR2 (FIG. 9). Therefore, CXCR4 is not directly involved in cAMP induction but cooperates in that regard with TLR2, which, on its own, induces a rather weak cAMP response (FIG. 9). That the synergistic C5a effect on cAMP induction actually involves a crosstalk with TLR2 was next shown.

Indeed, the ability of C5a to synergistically induce cAMP and activate PKA in P. gingivalis-stimulated wild-type macrophages was utterly absent in similarly stimulated Tlr2^{-/-} macrophages, which displayed only background activity levels (FIGS. 4C and 4D). However, the inherent capacity of Tlr2^{-/-} macrophages to elevate intracellular cAMP and activate PKA was confirmed by including a forskolin control (direct adenylate cyclase activator) (FIGS. 4C and 4D). This novel concept of C5aR-TLR2 crosstalk for synergistic cAMP-dependent PKA activation is consistent with additional findings from an in vivo experiment. Indeed, the PKA activity detected in freshly explanted peritoneal macrophages from P. gingivalis-infected mice was significantly reduced by TLR2 or C5aR deficiency, but not by TLR4 or C3aR deficiency, relative to cells from wild-type mice (FIG. 4E).

It was also shown that another synergistic interaction downstream of this receptor crosstalk involved PKA-dependent phosphorylation of glycogen synthase kinase-3β(GSK3β) on Ser9 (FIG. 4F), an event that inactivates this kinase which would otherwise positively regulate cell activation. Indeed, although C5a or P. gingivalis by themselves only slightly increased Ser9-phosphorylation of GSK3β, their combination displayed a synergistic effect which was inhibited by PKI 6-22 (but not by PD98059 control, an inhibitor of mitogen-activated protein kinase kinase) (FIG. 4F). Importantly, the GSK3β inhibitor SB216763 mimicked the inhibitory C5a effect on P. gingivalis-induced iNOS expression and nitric oxide production, as did 8-Br-cAMP (PKA agonist; positive control) (FIG. 4G). Thus, GSK3β appears to regulate iNOS and nitric oxide downstream of PKA in C5a plus P. gingivalis-challenged macrophages.

The C5aR-TLR2 crosstalk is also consistent with confocal microscopy findings revealing, for the first time, co-localization of the two receptors in P. gingivalis-stimulated macrophages (FIG. 4H), and with fluorescence resonance energy transfer (FRET) experiments indicating that C5aR, TLR2, and P. gingivalis come into molecular proximity (FIG. 4I). Indeed, FRET analysis revealed significant energy transfer between Cy3-labeled C5aR and Cy5-labeled TLR2 in P. gingivalis-stimulated but not resting macrophages (FIG. 4I). No significant energy transfer was detected between Cy3-labeled C5aR and Cy5-labeled TLR5 or MHC Class I (controls) under the same conditions (FIG. 4I). Moreover, significant energy transfer was observed between FITC-labeled P. gingivalis and TRITC-labeled C5aR or TLR2 (but not TLR5 or MHC Class I) (FIG. 4I). However, unlike TLR2, which can directly be engaged by P. gingivalis, C5aR appeared to associate indirectly with P. gingivalis in a TLR2-dependent way; indeed, the P. gingivalis-05aR FRET association was abrogated in Tlr2^{-/-} macrophages (FIG. 4I). Taken together, the findings from FIG. 4 firmly establish a crosstalk between C5aR and TLR2 for synergistic induction of cAMP signaling.

FRET analysis further revealed that, in P. gingivalis-challenged macrophages, C5aR also associates with CXCR4 (FIG. 4I), suggesting co-association of all three receptors (CXCR4, TLR2, C5aR). These interactions likely occur in lipid rafts since all three receptors (but not TLR5 or MHC Class I) come within FRET proximity with an established lipid raft marker (GM1 ganglioside) in P. gingivalis-stimulated macrophages, unless the rafts are disrupted by methyl-β-cyclodextrin (FIG. 10). Although the C5aR-TLR2 crosstalk can proceed independently of CXCR4 and potently up-regulate cAMP (FIG. 4B), maximal cAMP induction requires cooperation of all three receptors (FIG. 4K model).

### Example 14—Supplemental Material

Supplemental experiments demonstrated that C5a dose-dependently promotes the intracellular survival of P. gingivalis and the cAMP response. Peritoneal mouse macrophages were incubated with P. gingivalis in the presence of increasing concentrations of C5a, and viable counts of internalized bacteria at 24 hours post-infection were determined by CFU enumeration (FIG. 5A). In addition, P. gingivalis-induced cAMP responses in macrophages were assayed at 1 hour in the presence of increasing concentrations of C5a (FIG. 5B).

Supplemental experiments also demonstrated that C5a does not affect P. gingivalis phagocytosis. First, experiments were performed to determine the effect of C5a (50 nM) or C3a (200 nM) on P. gingivalis phagocytosis by unprimed or IFN-γ-primed mouse peritoneal macrophages (FIG. 6A). The phagocytic index was calculated following a 30-min incubation using the following formula: % positive cells for fluorescently labeled P. gingivalis×MFI/100 (extracellular fluorescence was quenched prior to flow cytometry). Mouse macrophages were incubated at 37° C. for 30 min (B) or 24 hours (C) with medium, C5a (50 nM) only, or P. gingivalis (MOI=10:1) with or without C5a (50 nM). The expression levels of the indicated receptors, which coordinately mediate P. gingivalis uptake, were determined by flow cytometry after cell staining with appropriate fluorescently labeled antibodies (FIGS. 6B and 6C). The 30-min time point was examined to determine possible induced surface expression of preformed receptors from intracellular pools. No significant differences were observed between the C5a in the presence of P. gingivalis and the P. gingivalis alone. Mouse-specific mAbs to TLR2 (clone 6C2), TLR1 (TR23), CD14 (Sa2-8), CD11b (M1/70), and CD18 (M18/2) were obtained from e-Bioscience.

Supplemental experiments also examined the relative expression of negative regulators of TLR signaling in P. gingivalis-stimulated macrophages in the absence or presence of C5a. Mouse macrophages were stimulated with P. gingivalis (Pg; at a MOI=10:1) or medium control, in the presence or absence of 50 nM of C5a, and incubated for 4 hours. Quantitative real-time PCR (ABI 7500 Fast System; Applied Biosystems) was used to determine mRNA expression levels for the indicated molecules (normalized against GAPDH mRNA levels), which are shown in FIG. 7. No significant differences were observed between C5a in the presence or absence of P. gingivalis.

C5a inhibits nitric oxide production in a dose- and time-dependent way. Mouse peritoneal macrophages were left untreated (FIGS. 8A, 8C, and 8E) or primed with 100 ng/ml IFN-gamma (FIGS. 8B, 8D, and 8F) overnight, washed, and incubated for 24 hours under the following conditions. In Panels A and B, the cells were incubated with P. gingivalis (Pg) in the presence of the indicated increasing concentrations of C5a. In Panels C and D, the cells were incubated with Pg with or without C5a (50 nM), which was added either together with the bacteria into the macrophage cultures (time “0”) or was delayed for various times, as indicated (“uninhibited control” denotes the absence of C5a throughout the experiment). In Panels E and F, the cells were incubated with Pg, with or without C5a (50 nM) for the indicated time intervals. Pg was used at a MOI=10:1 throughout and NO₂⁻ was assayed by the Griess reaction.

Supplemental experiments also were performed to examine TLR2-dependent cAMP production by P. gingivalis (FIG. 9). CHO-K1 cells, transfected with the indicated receptors (using expression plasmids from InVivogen and the PolyFect transfection reagent from Qiagen) were stimulated (or not) with P. gingivalis for 1 h and assayed for intracellular cAMP.

Supplemental experiments also examined the association of TLR2, C5aR, and CXCR4 with GM1 (lipid raft marker) in P. gingivalis-stimulated macrophages (FIG. 10). Mouse macrophages were pretreated (or not) with methyl-β-cyclodextrin (MCD; 10 mM for 30 min) and then stimulated for 10 min with P. gingivalis (Pg; MOI=10:1) or medium only (med). Fluorescence resonance energy transfer (FRET) between TLR2, C5aR, CXCR4, TLR5, or MHC Class I (Cy3-labeled) and the GM1 ganglioside (Cy5-labeled) was measured from the increase in donor (Cy3) fluorescence after acceptor (Cy5) photobleaching. TLR5 and MHC Class I served as negative controls. The indicated maximum (Max) and minimum (Min) FRET efficiencies in the system were determined, respectively, as the energy transfer between two different epitopes on the same molecule (TLR2) or between molecules that do not engage in heterotypic associations (TLR2 and MHC Class I). As expected, max FRET values (38±1.2) were not affected by the cell activation status (med vs. Pg) or the use or not of MCD.

Supplemental experiments also evaluated the generation of C5a by P. gingivalis from heat-inactivated human serum (FIG. 11). Heat-inactivated human serum was incubated with or without P. gingivalis (10⁸ bacterial cells per ml) for 30 min at 37° C., and C5a generation was determined using a Human C5a ELISA Kit (BD Biosciences). RESULTS???

Supplemental experiments also demonstrated the up-regulation of IL-6 production by C5a in P. gingivalis-stimulated macrophages (FIG. 12). Mouse peritoneal macrophages were incubated for 5 or 24 hours at 37° C. with P. gingivalis (Pg; MOI=10:1) in the presence or absence of C5a (50 nM), and culture supernatants were assayed for IL-6 by ELISA. P. gingivalis was detected in blood and internal organs of wild-type and C5aR-deficient (C5ar^{-/-}) mice after intraperitoneal infection. Twenty-four hours post-intraperitoneal infection with 5×10⁷ CFU, P. gingivalis bacterial loads were determined by plating serial dilutions of blood and tissue homogenates on blood agar plates subjected to anaerobic culture. Cultures were considered positive if at least three colonies of P. gingivalis were identified. Results are presented in Table 1.

TABLE 1

% mice with positive culture
(n = 5)

| Organs | wild-type | C5ar^{-/-} |
|---|---|---|
| Blood | 80 | 0 |
| Spleen | 0 | 0 |
| Kidney | 0 | 0 |
| Liver | 0 | 0 |
| Lungs | 0 | 0 |

### Part B: C5a Receptor Impairs IL-12-Dependent Clearance of Porphyromonas gingivalis and is Required for Induction of Periodontal Bone Loss

### Example 15—Reagents, Bacteria, and Mice

Mouse C5a and C5a^{desArg} were purchased from Cell Sciences or the R&D Systems. Mouse rIFN-γ, goat polyclonal anti-mouse IL-12 IgG, and anti-mouse IL-23 (p19) IgG were from R&D Systems. U0126 and wortmannin were purchased from the Cell Signaling Technology. The cyclic hexapeptide Ac-F[OP-dCha-WR] (acetylated phenylalanine-[ornithine-proline-D-cyclohexylalanine-tryptophan-arginine]), a specific and potent C5aR antagonist (also known as PMX-53) and the C3aR antagonist SB290157 were synthesized as previously described (Finch et al., 1999, J. Med. Chem., 42:1965-74; Markiewski et al., 2008, Nat. Immunol., 9:1224-35; Ames et al., 2001, J. Immunol., 166:6341-8). A8^{Δ71-73}, a dual antagonist of C5aR and C5a-like receptor-2, was expressed essentially as previously described (Otto et al., 2004, J. Biol. Chem., 279:142-51). Specifically, the A8^{Δ71-73} sequence was created by three cycles of mutagenesis of the original human C5a construct (Ritis et al., 2006, J. Immunol., 177:4794-802) using the QuickChange XL Site-Directed Mutagenesis Kit from Stratagene. The three pairs of complementary primers used for mutagenesis are as follows (forward sequences given): 1) 5′-GTT ACG ATG GAG CCG CCG TTA ATA ATG ATG-3′ (SEQ ID NO:1); 2) 5′-CCG TGC TAA TAT CTC TTT TAA ACG CAT GCA ATT GGG AAG G-3′ (SEQ ID NO:2); and 3) 5′-CTC TTT TAA ACG CTC GTG AAA GCT TAA TTA GC-3′ (SEQ ID NO:3), corresponding to mutations 1) C27A, 2) H67F and D69R, and 3) M70S and Δ(71-74), respectively. The protein was then expressed and purified as previously described (Ritis et al., 2006, J. Immunol., 177:4794-802). All reagents were used at optimal concentrations determined in preliminary or published studies by our laboratories. When appropriate, DMSO was included in medium controls and its final concentration was ≦0.2%.

P. gingivalis ATCC 33277 and its isogenic KDP128 mutant, which is deficient in all three gingipain genes (rgpA, rgpB, and kgp) (Grenier et al., 2003, Infect. Immun., 71:4742-8), kindly provided by Dr. K. Nakayama, Nagasaki University, Japan, were grown anaerobically from frozen stocks on modified Gifu anaerobic medium-based blood agar plates for 5-6 days at 37° C., followed by anaerobic subculturing for 18-24 hours at 37° C. in modified Gifu anaerobic medium broth (Nissui Pharmaceutical).

Thioglycollate-elicited macrophages were isolated from the peritoneal cavity of wild-type or mice deficient in TLR2 or C5aR (Hajishengallis et al., 2006, Cell. Microbiol., 8:1557-70; Zhang et al., 2007, Blood, 110:228-36) in compliance with established institutional policies and federal guidelines. Both BALB/c and C57BL/6 C5aR-deficient mice were used (with their respective wild-type controls): The BALB/c mice were obtained from The Jackson Laboratory; and the C57BL/6 C5aR-deficient mice were originally provided by Dr. Craig Gerard (Harvard Medical School) and are now housed at The Jackson Laboratory. The TLR2-deficient mice were originally C57BL/6 (The Jackson Laboratory) and were backcrossed for nine generations onto a BALB/c genetic background before their use in these studies. The macrophages were cultured at 37° C. and 5% CO₂ in RPMI 1640 (InVitrogen) supplemented with 10% heat-inactivated FBS, 2 mM L-glutamine, 100 units/ml penicillin G, 100 μg/ml streptomycin, and 0.05 mM 2-ME. None of the experimental treatments affected cell viability (monitored by the CellTiter-Blue™ assay; Promega) compared to medium-only treatments.

### Example 16—Cell Activation Assays

Induction of nitric oxide production was assessed by measuring the amount of NO₂⁻ (stable metabolite of nitric oxide) in stimulated culture supernatants using a Griess reaction-based assay kit (R&D Systems), as previously performed (Hajishengallis et al., 2008, PNAS USA, 105:13532-7). Levels of cAMP in activated cell extracts were measured using a cAMP enzyme immunoassay kit (Cayman Chemical) (Liang et al., 2007, J. Immunol., 178:4811-9). C5a-induced intracellular calcium mobilization was monitored in cells (4×10⁶) loaded with 1 μM Indo 1-AM in the presence of 1 μM pluronic acid, as previously described (Ali et al., 1993, J. Biol. Chem., 268:24247-54). Calcium traces were recorded in a Perkin-Elmer fluorescence spectrometer (Model 650-19) with an excitation wavelength of 355 nm and an emission wavelength of 405 nm. Induction of cytokine production in activated cell culture supernatants or in the peritoneal fluid of infected mice was determined by ELISA using kits from eBioscience or Cell Sciences. C5a levels were measured by sandwich ELISA, employing a pair of capture and biotinylated anti-05a mAbs (BD Pharmingen), according to the manufacturer's protocol.

### Example 17—Intracellular Killing Assay

The viability of phagocytosed P. gingivalis was monitored by an antibiotic protection-based intracellular survival assay, as previously described (Wang et al., 2007, J. Immunol., 179:2349-58)). Briefly, mouse peritoneal macrophages were allowed to phagocytose P. gingivalis (at a MOI=10:1; 5×10⁶ bacteria and 5×10⁵ macrophages) for 30 min at 37° C. This was followed by washing to remove extracellular nonadherent bacteria and 1-hour treatment with antibiotics (300 μg/ml gentamicin and 200 μg/ml metronidazole) to eliminate residual or extracellular adherent bacteria. The macrophages were subsequently cultured overnight for a total of 24 hours. Immediately after, the macrophages were washed and lysed in sterile distilled water and viable counts of internalized P. gingivalis were determined by plating serial dilutions of macrophage lysates on blood agar plates subjected to anaerobic culture.

### Example 18—In Vivo Mouse Studies

I.p. Challenge Model.

10-12 week-old mice were infected i.p. with P. gingivalis (5×10⁷ CFU) and sampled by peritoneal lavage to measure production of cytokines and enumerate recovered CFU (following anaerobic growth on blood agar plates) (Hajishengallis et al., 2008, PNAS USA, 105:13532-7), as detailed in the respective figure description.

P. gingivalis-Induced Bone Loss.

The P. gingivalis-induced periodontal bone loss model was used essentially as originally described (Baker et al., 2000, Infect. Immun., 68:5864-8) with slight modifications as was previously described (Wang et al., 2007, J. Immunol., 179:2349-58). Briefly, upon suppression of the normal oral flora with antibiotics, 10-12 week-old wild-type mice or mice deficient in C5aR or TLR2 were infected by oral gavage five times at 2-day intervals with 10⁹ CFU P. gingivalis suspended in 2% carboxymethylcellulose. Sham-infected controls received 2% carboxymethylcellulose alone. The mice were euthanized six weeks later and assessment of periodontal bone loss in defleshed maxillae was performed under a dissecting microscope (×40) fitted with a video image marker measurement system (VIA-170K; Fryer). Specifically, the distance from the cementoenamel junction (CEJ) to the alveolar bone crest (ABC) was measured on 14 predetermined points on the buccal surfaces of the maxillary molars. To calculate bone loss, the 14-site total CEJ-ABC distance for each mouse was subtracted from the mean CEJ-ABC distance of sham-infected mice. The results were expressed in mm and negative values indicate bone loss relative to sham-infected controls.

Age-Associated Periodontal Bone Loss.

Aging mice develop naturally occurring inflammatory periodontal bone loss, which becomes quite dramatic after 9 months of age. To determine the role of C5aR in periodontal bone loss in this chronic model, C5aR-deficient and wild-type controls were raised in parallel and bone loss was assessed as described above when the mice became 16-month old.

All animal procedures were approved by the Institutional Animal Care and Use Committee, in compliance with established Federal and State policies.

### Example 19—Statistical Analysis

Data were evaluated by analysis of variance and the Dunnett multiple-comparison test using the InStat program (GraphPad Software, San Diego, Calif.). Where appropriate (comparison of two groups only), two-tailed t tests were performed. P<0.05 was taken as the level of significance.

### Example 20—P. gingivalis Proactively and Selectively Inhibits IL-12p70 Production Via C5aR-TLR2 Crosstalk

Whether C5a inhibits P. gingivalis-induced IL-12p70 in peritoneal macrophages was investigated. Since macrophages are generally poor producers of IL-12p70 in vitro unless primed with IFN-gamma, macrophages used in these experiments were primed with IFN-gamma (0.1 μg/ml). E. coli LPS-stimulated macrophages were used as a control since C5a has been shown to inhibit IL-12p70 through a C5a/C5aR-LPS/TLR4 crosstalk. The host TLR response against P. gingivalis is predominantly mediated by TLR2, both in vitro and in vivo. Therefore, whether C5a-mediated inhibition of P. gingivalis-induced IL-12p70 could involve a C5aR-TLR2 crosstalk additionally was examined. It was found that the abilities of both P. gingivalis and LPS to induce IL-12p70 production were significantly inhibited by C5a (p<0.01; FIG. 13A). These inhibitory effects were specifically mediated by C5aR signaling, since they were completely reversed by a specific C5aR antagonist (C5aRA) (p<0.01; FIG. 13A).

Intriguingly, however, C5aRA significantly enhanced the induction of IL-12p70 production, even in P. gingivalis-stimulated macrophages that were not treated with exogenous C5a (p<0.01; FIG. 13A); this up-regulatory effect was not seen in C5a-untreated and LPS-stimulated macrophages (FIG. 13A). It was previously shown that P. gingivalis generates C5a in complement-inactivated serum, and the results described herein have now confirmed the presence of C5a in the supernatants of wild-type P. gingivalis-treated cells (1460±246 pg/ml, n=3); in contrast, C5a in the supernatants of KDP128-treated cells was below the assay detection limit (<39 pg/ml). Therefore, endogenously-generated C5a limits P. gingivalis-induced IL-12p70 production, which is thus enhanced in the presence of C5aRA. This notion was substantiated by the finding that KDP128 failed to regulate IL-12p70, unless exogenous C5a was added in the cell cultures (FIG. 13B). Indeed, C5aRA had no effect on KDP128-induced IL-12p70 in the absence of exogenously added C5a (FIG. 13B). Interestingly, in the absence of exogenous treatments with C5a or C5aRA, KDP128 induced significantly higher levels of IL-12p70 than wild-type P. gingivalis (p<0.05; FIG. 13B). This is attributed to the inability of KDP128 to generate C5a in the culture supernatants that would limit IL-12p70 production. The ability of P. gingivalis to induce IL-12p70 was completely abrogated in TLR2-deficient macrophages, whereas, as expected, LPS-induced IL-12p70 was unaffected (FIG. 13C). Taken together, these data indicate that P. gingivalis activates a C5aR-TLR2 crosstalk, which inhibits IL-12p70 production in macrophages.

The C5aR crosstalk pathways with TLR2 or TLR4 for IL-12p70 regulation appear to be similar, since the inhibitory effects of C5a were abrogated by treatment with the MEK1/2-specific inhibitor U0126 but not by the PI3K inhibitor wortmannin (p<0.01; FIG. 13D). This implicates the MEK-ERK1/2 pathway in C5aR-mediated regulation of both TLR2- and TLR4-induced IL-12p70. On the other hand, the PI3K pathway is minimally involved, if at all. In the absence of C5a, however, wortmannin up-regulated LPS-induced IL-12p70 (p<0.01; FIG. 13D), suggesting that, under these conditions (lack of C5aR activation), PI3K can inhibit IL-12p70, as previously shown. The finding that wortmannin failed to regulate P. gingivalis-induced IL-12p70 (FIG. 13D) is likely attributed to the presence of endogenously produced C5a in the culture supernatants. On the other hand, U0126 appeared to up-regulate both LPS- and P. gingivalis-induced IL-12p70, but this difference reached statistical significance only for the latter (p<0.01; FIG. 13D). In summary, C5a-induced inhibition of IL-12p70 by P. gingivalis or LPS is mediated by ERK1/2 but not PI3K signaling, although PI3K can regulate LPS-induced IL-12p70 in the absence of C5aR activation.

The C5aR-dependent inhibition of IL-12p70 in P. gingivalis-stimulated macrophages was selective for this cytokine, since other pro-inflammatory cytokines (e.g., IL-6 and TNF-α) were actually up-regulated (p<0.01; FIG. 13E). These results indicate that P. gingivalis proactively and selectively inhibits IL-12p70 production by activating a C5aR-TLR2 crosstalk without requirement for immunological mechanisms of complement activation.

### Example 21—P. gingivalis Disables Human Neutrophils

Using the ‘chamber’ model, bacteria were injected into the lumen of a subcutaneously implanted titanium-coil chamber such that bacterial interactions with recruited inflammatory cells can be assessed accurately and quantitatively (Burns et al., 2006, J. Immunol., 177:8296-8300; Genco et al., 1991, Infect. Immun., 59:1255-63; and Graves et al., 2008, J. Clin. Periodontol., 35:89-105). The overwhelming majority of cells recruited into P. gingivalis (Pg)-injected chambers 24 h post-infection (>97%) were neutrophils. Moreover, since the host response against Pg was critically dependent on TLR2 (Burns et al., 2006, J. Immunol., 177:8296-8300; and Hajishengallis et al., 2008, J. Immunol., 181:4141-49), it was confirmed that TLR2 is expressed at normal levels in C5aR−/− mice. It was found that Pg also can undermine the killing function of neutrophils in a C5aR-dependent manner. Indeed, the aspirated chamber fluid from C5aR−/− mice 24 h post-infection contained significantly lower Pg CFU compared to wild-type mice (>95% reduction). Consistent with this, treatment of wild-type mice with PMX-53, a potent C5aR antagonist (C5aRA), but not an inactive analog, also reduced Pg viable counts.

To directly implicate neutrophils in this evasion mechanism, in vitro experiments were performed. It was shown that the ability of mouse or human neutrophils (purified from peripheral blood5 collected under IRB approval) to kill Pg was inhibited in the presence of C5a in a C5aR-dependent manner, whereas their oxidative burst response was enhanced.

These findings with human neutrophils are significant in that they demonstrate this mechanism in human cells, and they also demonstrate that Pg exploits C5aR signaling to evade killing by neutrophils, which still maintain their destructive oxidative and inflammatory responses.

### Example 22—C5aR Signaling In Vivo Differentially Regulates P. gingivalis-Induced Cytokine Responses

The biological significance of the C5aR-mediated inhibition of IL-12p70 production was next investigated. First, it was essential to determine whether C5aR signaling can regulate P. gingivalis-induced IL-12p70 production in vivo. For this purpose, wild-type mice were i.p.-administered C5aRA followed by i.p. challenge with P. gingivalis. Mice deficient in C5aR or TLR2 were similarly challenged with P. gingivalis, and all mice were sampled 5 h post-infection by peritoneal lavage. In addition to IL-12p70, production of IFN-gamma (which is positively regulated by IL-12p70), IL-23 (an IL-12 family cytokine which shares a common IL-12/IL-23p40 subunit with IL-12p70), as well as proinflammatory cytokines (which have been implicated in inflammatory bone resorption in periodontitis (IL-1beta, IL-6, and TNF-alpha)) was determined. C5aRA-treated wild-type mice and C5aR-deficient mice elicited significantly higher levels of IL-12p70, IFN-gamma, and IL-23 compared to PBS-treated wild-type controls (p<0.01-0.05; FIG. 14). In contrast, the induction of IL-1beta, IL-6, and TNF-alpha production was inhibited by C5aR blockade or C5aR deficiency (p<0.01; FIG. 14). On the other hand, the induction of all tested cytokines was abrogated in TLR2-deficient mice (p<0.01; FIG. 14). None of these cytokines was detectable in the peritoneal fluid of mice not challenged with P. gingivalis. These data show that C5aR signaling in vivo selectively inhibits the ability of P. gingivalis to induce TLR2-dependent IL-12 family cytokines (IL-12p70 and IL-23). The observed down-regulation of IFN-gamma is most likely secondary to inhibition of IL-12p70 production. On the other hand, maximal induction of IL-1beta, IL-6, and TNF-alpha requires intact signaling by both C5aR and TLR2.

### Example 23—C5aR-Mediated Inhibition of IL-12p70 Promotes P. gingivalis Survival In Vivo

Whether the C5aR-mediated inhibitory effect on IL-12p70 production (FIG. 14) is exploited by P. gingivalis was addressed in subsequent experiments. Wild-type mice were i.p.-treated with C5aRA (or PBS control) and infected with P. gingivalis by the same route. The C5aRA-treated mice comprised several groups, including mice given anti-IL-12 IgG, anti-IL-23p19 IgG, or non-immune IgG control. Treatment with anti-IL-23p19 was included because the anti-IL-12 Ab reacts with both IL-12p70 subunits, p35 and p40, the latter of which is shared by the heterodimeric IL-23 (IL-12/IL-23p40 and IL-23p19). Thus, the experiment was designed in a way that would allow specific implication of IL-12p70 or both IL-12p70 and IL-23 (or none) in P. gingivalis immune clearance. At 24 h post-infection, the peritoneal lavage fluid from C5aRA-treated mice contained about 2 log₁₀ units less P. gingivalis CFU compared to mice pretreated with PBS control (p<0.01; FIG. 15A). However, the enhanced ability of C5aRA-treated mice to clear P. gingivalis was significantly (p<0.01) counteracted by anti-IL-12 treatment, though not by anti-IL-23p19 or non-immune IgG (FIG. 15A). Viable P. gingivalis CFU counts were not detected in the blood or in homogenates of several organs examined (spleen, kidney, liver, and lungs) from any of the mouse groups. Taken together with the FIG. 14 results, these data show that C5aR signaling inhibits IL-12p70 production and this inhibitory effect is exploited by P. gingivalis to resist immune clearance. This conclusion was further substantiated by similar findings from a related experiment in which C5aRA-treated mice were replaced by C5aR-deficient mice (FIG. 14B).

In a side-by-side comparison of the in vivo survival capacities of wild-type P. gingivalis and KDP128, the mutant was recovered at significantly lower levels (>500-fold difference compared to wild-type P. gingivalis) from the peritoneal cavity of wild-type mice (p<0.01; FIG. 15C). This difference in survival capacity may be related, at least in part, to the inability of KDP128 to generate C5a, as shown in vitro. Even in vivo, where physiological mechanisms (e.g., activation of the complement cascade) could contribute to C5a generation, the peritoneal fluid of KDP128-infected mice contained significantly lower levels of C5a (374±93 pg/ml) than that of wild-type P. gingivalis-infected mice (2174±513 pg/ml) (p<0.01; n=5 mice per group); C5a levels at baseline (uninfected mice) were 101±47 pg/ml. Consistent with these considerations, the survival of KDP128 was not significantly affected by C5aR deficiency (FIG. 15C), suggesting that the mutant cannot productively exploit C5aR to promote its survival, as the wild-type organism does. In conclusion, a great part of in vivo generated C5a can be attributed to the enzymatic action of P. gingivalis which thereby can efficiently manipulate IL-12p70 production and promote its survival.

### Example 24—Comparison of C5a and C5a^{desArg} in Regulating IL-12p70 and Other Macrophage Activities

C5a is relatively unstable in biological fluids and is rapidly converted to its desarginated form (C5a^{desArg}). In fact, a large part of in vivo detected C5a (see above) may represent C5a^{desArg} since the capturing antibody used in the sandwich ELISA (BD Pharmingen) recognizes a neoepitope exposed in both C5a or C5a^{desArg} (though not in intact C5). C5a^{desArg} does not have anaphylactic action but retains a number of other biological activities. Thus whether it shares the capacity of C5a to regulate IL-12p70 was investigated. It was found that C5a^{desArg} also can inhibit P. gingivalis-induced IL-12p70 production, though not as strongly as C5a. Specifically, C5a^{desArg} mediated significant (p<0.05) inhibition of IL-12p70 at 50 nM but not at 10 nM, at which concentration C5a was already effective (FIG. 16A). However, the increased stability and, thus, higher prevalence of C5a^{desArg} compared to intact C5a, suggests a possible significant role for the desarginated molecule in IL-12p70 regulation.

Although C5a^{dArg} also binds to the C5a-like receptor-2 (GPR77) with high affinity, its observed modulatory effect on IL-12p70 production was likely mediated via the C5aR (CD88). In this regard, C5aRA by itself caused full reversal of the inhibitory effect of C5a^{desArg}, whereas a dual C5aR/C5a-like receptor-2 antagonist (A8^{Δ71-73}) had a comparable effect (FIG. 16B). In contrast, the C3aR antagonist, SB290157, (control) did not influence the ability of C5a^{desArg} to inhibit induction of IL-12p70 by P. gingivalis (FIG. 16B).

C5a was previously implicated in synergistic interactions with P. gingivalis that elevate cAMP in macrophages, leading to inhibition of nitric oxide production and of intracellular killing. Whether these evasion mechanisms can also be activated by C5a^{desArg} was investigated. Side-by-side comparison revealed no significant differences between C5a and C5a^{desArg} when tested at 50 nM in elevating cAMP, inhibiting nitric oxide, and promoting its intracellular survival (FIG. 16, C-E). However, when the compounds were tested at 10 nM, C5a exhibited stronger effects than C5a^{desArg} (FIG. 16, C-E). In view of the strict dependence of C5a on intracellular Ca²⁺ mobilization to synergistically elevate cAMP, it was hypothesized that C5a^{desArg} could similarly induce intracellular Ca²⁺ responses. Indeed, at 50 nM, C5a and C5a^{desArg} induce dcomparable intracellular Ca²⁺ mobilization in macrophages (FIG. 17A), whereas only C5a was active in that regard in neutrophils (FIG. 17B). Taken together, the data from FIGS. 16 and 17 indicate that P. gingivalis can exploit C5a even after its conversion to C5a^{desArg} to undermine macrophage defense functions (induction of IL-12p70, activation of intracellular killing).

### Example 25—C5aR Mediates Periodontal Bone Loss

The involvement of C5aR signaling in P. gingivalis immune evasion and in the induction of pro-inflammatory cytokines (FIG. 13-16) such as IL-1beta, IL-6, and TNF-alpha that mediate periodontal bone resorption, suggested that C5aR may play an important role in P. gingivalis-induced periodontitis. Indeed, P. gingivalis failed to induce significant periodontal bone loss in C5aR-deficient BALB/c or C57BL/6 mice, in stark contrast to corresponding wild-type mice, which developed significant bone loss relative to sham-infected controls (p<0.01; FIGS. 18 A, B, and E). TLR2 participates in crosstalk interactions with C5aR that a) promote mechanisms of P. gingivalis immune evasion and b) induce production of bone-resorptive cytokines (FIG. 14). Sensibly, therefore, TLR2-deficient BALB/c mice were similarly shown to be resistant to P. gingivalis-induced periodontal bone loss (FIGS. 18 C and E).

Mice used for P. gingivalis-induced periodontitis studies are usually 8-12 week-old and sham-infected controls do not develop appreciable bone loss. However, aging mice, like aging humans, gradually develop naturally-occurring inflammatory periodontal bone loss (due to chronic exposure to indigenous periodontal bacteria), which becomes quite dramatic after 9 months of age. To determine the role of C5aR in the age-associated periodontitis model, C5aR-deficient BALB/c mice and wild-type controls were raised until the age of 16 months. It was found that old C5aR-deficient mice were significantly protected against age-associated periodontitis relative to similarly aged wild-type controls (p<0.01; FIG. 18D). Therefore, C5aR is involved in chronic, age-associated periodontal bone loss.

### Example 26—Conclusions

On the one hand, C5aR signaling inhibits TLR2-dependent IL-12p70 induction and interferes with immune clearance of P. gingivalis. On the other hand, the P. gingivalis-instigated C5aR-TLR2 crosstalk leads to up-regulation of other proinflammatory cytokines (e.g., IL-1beta, IL-6, and TNF-alpha). Therefore, this pathogen does not appear to cause a generalized immunosuppression but, rather, has evolved the ability to selectively target pathways that could result in its elimination. In fact, non-selective immunosuppression would not be advantageous to P. gingivalis; while such strategy could certainly protect P. gingivalis against host immunity, at the same time, the pathogen would be condemned to starvation. Indeed, P. gingivalis is an asaccharolytic organism with a strict requirement for peptides and hemin, and, thus, depends on the continuous flow of inflammatory serum exudate (gingival crevicular fluid) to obtain these essential nutrients and survive in its periodontal niche. Therefore, the proactive release of C5a by P. gingivalis and the ensuing C5a-induced inflammation, including increased vascular permeability and proinflammatory synergy with TLRs, can contribute to nutrient procurement. Moreover, the ability of P. gingivalis to induce C5aR-dependent periodontal bone loss expands the useful space for increased niche for the pathogen.

Based on the results herein, it becomes apparent that P. gingivalis uses a quite antithetical strategy relative to, for example, Staphylococcus aureus, which promotes its survival by actually blocking C5a binding and C5aR activation via a secreted protein. This mechanism inhibits C5a-induced inflammation and phagocytic cell chemotaxis, and protects S. aureus from neutrophils and macrophages. On the other hand, the protozoan parasite, Leishmania major, exploits C5aR to evade host immunity but has to rely on C5a generation by the physiological complement cascade to be able to do so.

P. gingivalis-induced inflammation via the C5aR-TLR2 crosstalk may have important implications from a clinical perspective, since it is likely to cause collateral tissue damage (inflammatory periodontal bone destruction). This notion is supported by the findings herein that mice deficient in C5aR or TLR2 are both resistant to P. gingivalis-induced periodontitis. The fact that induction of bone loss is essentially absent in the absence of either C5aR or TLR2 signaling, argues against the possibility that C5aR and TLR2 contribute to periodontal pathogenesis through independent effector mechanisms. In this regard, both receptors are under P. gingivalis control and are induced to crosstalk, while in physical proximity, cooperatively leading to immune evasion and induction of inflammatory/bone-resorptive cytokines.

The C5a anaphylatoxin as well as the C3a anaphylatoxin are readily metabolized in serum and lose their C-terminal Arg due to carboxypeptidase activity. The resulting C3a fragment (C3a^{desArg}) is biologically inert in terms of C3a receptor-dependent functions, but retains antimicrobial activity which is exerted independent of the receptor. On the other hand, C5a^{desArg} can still bind C5aR, albeit with a lower affinity and a different mode of interaction relative to intact C5a. Although C5a^{desArg} is devoid of C5a anaphylactic (spasmogenic) activity, it retains other C5a activities to varying degrees depending on function and cell type involved. For example, monocytes and macrophages do not appear to distinguish between C5a and C5a^{desArg} in terms of induction of chemotaxis or lysosomal enzyme release, whereas neutrophils do. Thus, C5a^{desarg} retains the ability to inhibit P. gingivalis-induced IL-12p70 and nitric oxide production.

The results disclosed herein demonstrate that P. gingivalis has evolved to not only endure the host response by, for example, selectively suppressing critical ‘killing’ pathways, such as IL-12-dependent clearance, but also to benefit from the inflammatory response, while at the same time contributing to periodontal pathogenesis. The ability of P. gingivalis to inhibit innate immune functions via C5aR exploitation may also allow bystander bacteria, i.e., co-habiting the same niche, to evade immune control. In this context, P. gingivalis is thought of as a keystone periodontal species that could promote the survival and virulence of the entire microbial community. As such, preventing, reducing, or eliminating P. gingivalis via disruption of the mechanisms described herein may allow the prevention or treatment of periodontitis or diseases associated with periodontitis.

### Example 27—In Vivo Experiments

Experiments were performed to determine an effective dose of C5aRA (PMX-53) that inhibits periodontal inflammatory responses. Briefly, 0.1, 1, or 10 μg C5aRA (or a PBS control) were administered through 1-μl microinjections (using a 28.5-gauge MicroFine needle) on the mesial of the first molar and in the papillae between first and second and third molars, on both sides of the maxilla. These treatments were repeated five times at two day-intervals Immediately following each treatment, the mice were infected orally with Pg in 2% carboxymethylcellulose vehicle (or vehicle only). One week after the last infection, the gingiva were dissected and analyzed by real-time quantitative PCR for mRNA expression of IL-1beta and TNF-alpha (selected as the most typically involved in destructive periodontal inflammation). A C5aRA dose of 1 μg was highly effective in inhibiting induction of both IL-1 beta and TNF-alpha and its efficacy were not significantly different from a 10-fold higher dose (FIG. 19A).

Because the antagonist was applied before each Pg infection treatment, this approach was considered preventive. In addition, however, it was determined if C5aRA acts in a therapeutic way (i.e., applied after infection and inflammation occurs). In this case, five oral infections with Pg were first performed, 2 weeks was allowed to pass (e.g., the time required to observe significant bone loss) and then 1 μg C5aRA (or equal amount of an inactive peptide analog or PBS) was applied twice weekly for a total of four times. The mice were euthanized three days after the last treatment. C5aRA, but not the inactive analog, significantly reversed induction of IL-1beta and TNF-alpha (FIG. 19B).

### OTHER EMBODIMENTS

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

We claim:

1. A method of treating or preventing periodontitis or diseases associated with periodontitis in an individual, comprising: (a) identifying an individual suffering from or at risk of developing periondontitis or a disease associated with periodontitis; and (b) administering to the individual a complement inhibitor selected from C1 Inhibitor (C1-inh), Factor H, a Factor D inhibitor, or any combination thereof, thereby treating or preventing the periodontitis or diseases associated with periodontitis.

2. The method of claim 1, wherein the complement inhibitor is C1-inh.

3. The method of claim 1, wherein the complement inhibitor is Factor H.

4. The method of claim 1, wherein the complement inhibitor is a Factor D inhibitor.

5. The method of claim 1, wherein the diseases associated with periodontitis are selected from the group consisting of atherosclerosis, diabetes, osteoporosis, and pre-term labor.

6. The method of claim 1, wherein the compound is administered by a method selected from parenteral, intradermal, subcutaneous, oral, nasal, topical, transdermal or transmucosal.

7. The method of claim 6, wherein the compound is administered to the periodontal pocket of the individual.

8. A method of reducing the amount of Porphyromonas gingivalis and/or the inflammation caused by P. gingivalis in an individual, comprising: (a) identifying an individual in which reduction in the amount of P. gingivalis is needed or desired, and (b) administering to the individual a complement inhibitor selected from C1 Inhibitor (C1-inh), Factor H, a Factor D inhibitor, or any combination thereof, thereby reducing the amount of P. gingivalis in the individual.

9. The method of claim 8, wherein the compound is C1-inh.

10. The method of claim 8, wherein the complement inhibitor is Factor H.

11. The method of claim 8, wherein the complement inhibitor is a Factor D inhibitor.

12. The method of claim 8, wherein the compound is administered by a method selected from parenteral, intradermal, subcutaneous, oral, nasal, topical, transdermal or transmucosal.

13. The method of claim 12, wherein the compound is administered to the periodontal pocket of the individual.
